(19) United States
(12) Patent Application Publication
(10) Pub. No.: US 2026/0248758 A1
(43) Pub. Date: Aug. 27, 2026

(54) LOW VISCOSITY PHARMACEUTICAL COMPOSITIONS CONTAINING MYCOPHENOLIC ACID FOR THE TREATMENT OF OCULAR DISORDERS

(71) Applicant: SURFACE OPHTHALMICS, INC., Pleasanton, CA (US)

(72) Inventors: Richard L. Lindstrom, Wayzata, MN (US); Kamran Hosseini, Pleasanton, CA (US)

(21) Appl. No.: 19/650,172

(22) Filed: Apr. 16, 2026

Related U.S. Application Data
(63) Continuation-in-part of application No. 18/100,505, filed on Jan. 23, 2023, now abandoned.
Application No. 18/100,505 is a continuation-in-part of application No. 17/582,627, filed on Jan. 24, 2022, now abandoned.
Application No. 17/582,627 is a continuation-in-part of application No. PCT/US2021/053552, filed on Oct. 5, 2021.
Application No. 18/100,505 is a continuation-in-part of application No. PCT/US2021/053552, filed on Oct. 5, 2021.
Application No. PCT/US2021/053552 is a continuation-in-part of application No. 16/650,071, filed on Sep. 21, 2018, now Pat. No. 11,766,421.
Application No. 18/100,505 is a continuation-in-part of application No. 17/418,813, filed on Dec. 4, 2019, now Pat. No. 12,070,442.
(60) Provisional application No. 62/562,809, filed on Sep. 25, 2017.
(60) Provisional application No. 62/785,312, filed on Dec. 27, 2018.
(60) Provisional application No. 63/088,850, filed on Oct. 7, 2020.
(60) Provisional application No. 63/317,770, filed on Mar. 8, 2022.
(60) Provisional application No. 63/402,704, filed on Aug. 31, 2022.
(60) Provisional application No. 63/415,419, filed on Oct. 12, 2022.

(51) Int. Cl. A61K 31/366; A61K 9/00; A61K 31/661; A61K 31/726; A61K 47/02; A61K 47/10; A61K 47/12; A61K 47/36; A61P 27/14

(52) U.S. Cl. CPC A61K 31/366; A61K 9/0048; A61K 31/661; A61K 31/726; A61K 47/02; A61K 47/10; A61K 47/12; A61K 47/36; A61P 27/14

(57) ABSTRACT

Low viscosity pharmaceutical compositions and methods for treating ocular disorders in a subject, which include mycophenolic acid or a pharmaceutically acceptable salt thereof at a concentration of 0.05% wt/wt to 0.30% wt/wt, a glucosaminoglycan at a concentration of 0.1% to 2.5% wt/wt, and optionally a betamethasone at a concentration of 0.01% to 0.08% wt/wt.

### CROSS REFERENCE TO RELATED APPLICATIONS

This is a continuation in part of U.S. patent application Ser. No. 18/100,505, filed Jan. 23, 2023, which is a continuation in part of U.S. patent application Ser. No. 17/582,627, filed Jan. 24, 2022, now abandoned, which is a continuation in part of U.S. patent application Ser. No. 16/650,071, filed Mar. 24, 2020, now U.S. Pat. No. 11,766,421, which is a US national phase of international patent application no PCT/US2018/052158, filed Sep. 21, 2018, now expired, which claims priority to U.S. provisional application No. 62/562,809, filed Sep. 25, 2017, now expired; the entire content of each application referenced in this paragraph is herein incorporated by reference in its entirety.

U.S. Ser. No. 18/100,505 is also a continuation in part of U.S. patent application Ser. No. 17/418,813, now U.S. Pat. No. 12,070,442, which is a US national phase application of international patent application no. PCT/US2019/064371, filed Dec. 4, 2019, now expired, which claims priority to U.S. provisional application No. 62/785,312, filed Dec. 27, 2018, now expired; the entire content of each application referenced in this paragraph is herein incorporated by reference in its entirety.

U.S. Ser. No. 18/100,505 is also a continuation in part of international patent application no. PCT/US2021/053552, filed Oct. 8, 2021, now expired, which claims priority to U.S. provisional application No. 63/088,850, filed Oct. 7, 2020, now expired; the entire content of each application referenced in this paragraph is herein incorporated by reference in its entirety.

U.S. Ser. No. 18/100,505 also claims priority to U.S. provisional patent application No. 63/317,770, filed Mar. 8, 2022, now expired, and U.S. provisional patent application No. 63/402,704, filed Aug. 31, 2022, now expired, and U.S. provisional patent application No. 63/415,419, filed Oct. 12, 2022, now expired; the entire content of each application referenced in this paragraph is herein incorporated by reference in its entirety.

### TECHNICAL FIELD

The invention relates to pharmaceutical compositions for the treatment of ocular disorders and more specifically to pharmaceutical compositions having a low viscosity and containing a low dose of mycophenolic acid or a pharmaceutically acceptable salt thereof (e.g. mycophenolate sodium), a low dose of a glycosaminoglycan, and optionally a low dose of betamethasone or a pharmaceutically acceptable salt thereof (e.g. betamethasone sodium phosphate) for use in the treatment of ocular disorders, such as but not limited to dry eye disease, blepharitis, meibomian gland disease (MGD), hyperemia, uveitis, and for use as treatment for ocular injury, such as after undergoing ocular surgery.

### BACKGROUND OF THE INVENTION

Ocular disorders are those that affect the health of the eye. Some of the more common ocular disorders include ocular surface disease, which indicates damage to the surface layers of the eye, namely, the cornea and conjunctiva. Among the more common ocular surface diseases are dry eye disease, blepharitis, and meibomian gland disease (MGD), which can be a precursor to blepharitis and/or dry eye disease and acute and perennial ocular allergy. Many patients manifest two or more of these diagnoses.

The American Academy of Ophthalmologists (AAO), in a Preferred Practice Pattern report, has defined dry eye syndrome/dry eye disease (DED) as a group of disorders of the tear film due to reduced tear production or tear film instability, associated with ocular discomfort and/or visual symptoms and inflammatory disease of the ocular surface. AA0 (2018). The ocular surface discomfort is often described as a feeling of dryness, burning, itchiness, or a sandy/gritty sensation. Baudouin (2014). A subset of patients with DED have primary Sjögren syndrome, an autoimmune disease mainly affecting the exocrine glands causing a sicca syndrome (dryness of the exocrine glands, particularly the eyes and mouth). The prevalence of clinically diagnosed dry eye is 0.4% to 0.5% over all ages and is highest among women and the elderly (65 years and older). AAO (2018).

The ocular surface and tear-secreting glands function as an integrated unit. Any alteration in tear film composition or volume leads to tear film instability, which results in ocular irritation and symptoms and possible damage to the ocular surface epithelium. Stern (2014), Baudouin (2014), Hyon (2014). Dysfunction of this integrated unit may develop from aging, a decrease in supportive factors (such as androgen hormones), systemic inflammatory diseases (such as Sjögren syndrome or rheumatoid arthritis), ocular surface diseases (such as herpes simplex virus (HSV) keratitis) or surgeries that disrupt the trigeminal afferent sensory nerves (e.g., LASIK), and systemic diseases or medications that disrupt the efferent cholinergic nerves that stimulate tear secretion. AAO (2018).

One core mechanism of DED is evaporation-induced tear hyperosmolarity, which is a hallmark of the disease. It damages the ocular surface both directly and by initiating inflammation. Craig (2017). Two broad forms of DED are recognized, aqueous deficient dry eye (ADDE) and evaporative dry eye (EDE). Baltatzis (2003).

In ADDE, tear hyperosmolarity results when lacrimal secretion is reduced, in conditions of normal evaporation from the eye. In the Western world the most common cause of ADDE is inflammatory infiltration of the lacrimal gland, encountered most severely in the DED associated with autoimmune disorders such as Sjögren syndrome and, with lesser severity, in non-Sjögren syndrome.

In EDE, tear hyperosmolarity is caused by excessive evaporation from the exposed tear film in the presence of a normally functioning lacrimal gland. Since tear osmolarity is a function of tear evaporation in either ADDE or EDE, tear hyperosmolarity arises due to evaporation from the ocular surface and, in that sense, all forms of DED are evaporative. In other words, EDE is more accurately considered a hyper-evaporative state.

Current DED treatments include the use of artificial tears (topical lubricant preparations), topical corticosteroids, punctal plugs, ophthalmic immune modulators, Lymphocyte function-associated antigen 1 (LFA-1) antagonists, and devices and drugs that stimulate lacrimation.

Artificial tears are the most common treatment strategy for patients with DED of all severities. Jones (2017). Most clinical trials involving topical lubricant preparations have documented some improvement (but not resolution) of subjective symptoms and improvement in some objective parameters. However, the improvements noted are not necessarily any better than those seen with the vehicle or other non-preserved artificial lubricants. The elimination of preservatives and the development of newer, less toxic preservatives have made ocular lubricants better tolerated by dry eye patients.

Although considered effective, topical corticosteroid anti-inflammatory products (such as prednisolone acetate 1.0% ophthalmic suspension (OMNIPRED), difluprednate 0.05% ophthalmic emulsion (DUREZOL), prednisolone acetate 1.0% ophthalmic suspension (PREDFORTE) are not approved for the treatment of DED. This class of medicines is generally not recommended for long-term use due to the substantial associated risk of side effects such as glaucoma, cataract, infection, and wound-healing delay, but is sometimes used off-label short term (in pulses) for treatment of DED. Omnipred PI (2006), PredForte PI (2018). An exception is loteprednol etabonate 0.25% (EYSUVIS), which was approved in 2020 by the Food and Drug Administration (FDA) for short-term treatment of the signs and symptoms of dry eye syndrome (DES).

Punctal occlusion is a non-pharmacological intervention for dry eye when artificial tears or other remedies do not effectively ameliorate symptoms. The evidence in the systematic review for punctal plug trials suggests that improvements in symptoms and commonly tested dry eye signs are inconclusive. Ervin (2018).

The RESTASIS cyclosporine ophthalmic emulsion 0.05% is a calcineurin inhibitor immunosuppressant indicated to increase tear production in patients whose tear production is presumed to be suppressed due to ocular inflammation associated with keratoconjunctivitis sicca (KCS). RESTASIS PI (2017). The most common adverse reaction noted in clinical trials following the use of RESTASIS was ocular burning (17%). Lifitegrast ophthalmic solution 5% (XIIDRA) is a lymphocyte function-associated antigen-1 (LFA-1) antagonist indicated for the treatment of the signs and symptoms of DED. XIIDRA PI (2016). The exact mechanism of action of lifitegrast in DED is not known. The most common adverse reactions reported in 5% to 25% of clinical trial patients were instillation site irritation, dysgeusia and reduced visual acuity.

Mechanical trigeminal nerve stimulation is another approach that has been used in the past to increase tear production, which includes using micro-electric pulses. This was the approach pursued in the TRUE TEAR device, which was approved by the FDA to achieve that goal, but it has since been discontinued. The parasympathetic nervous system (PNS), via the trigeminal parasympathetic pathway, controls tear film homeostasis by innervating the lacrimal functional unit (LFU), which includes the cornea, conjunctiva, lacrimal glands, meibomian glands and goblet cells. In addition, there has been other pharmacological agent(s) such as varenicline solution nasal spray 0.03 mg (TYRVAYA) that were approved to stimulate tear production through similar pathways. However, adverse effects found with the use of TYRVAYA include sneezing, coughing, throat irritation and nose irritation.

Accordingly, there remains a need for new therapeutics for the treatment of DED and other ocular disorders without the above-described adverse events.

### BRIEF SUMMARY OF THE INVENTION

The invention is directed to therapeutic compositions and their use for the treatment and prevention of ocular disorders, such as ocular disease and injury to the surface of the eye. Among the improvements herein include the improved relief of ocular disorders signs and symptoms in instances where mycophenolic acid, betamethasone, or both mycophenolic acid and betamethasone are administered. Further, the above is accomplished in different embodiments using one or more of: a surprisingly low dosage of mycophenolic acid, a surprisingly low dosage of betamethasone, a surprisingly low dosage of a glycosaminoglycan (eg. Chondroitin sulfate), a surprising short, pulsed treatment period, and a synergistic therapeutic effect that occurs when administering a combination of betamethasone and mycophenolic acid. Each of the compositions have a low viscosity even in the presence of glucosaminoglycan(s) and other agents known to increase viscosity. Clinical studies confirmed that each of these treatments was not only effective for treating signs and symptoms of ocular surface disease, but they also lacked the adverse effects associated with conventional steroid-based treatments. Among the disorders that can be treated include ocular surface diseases such as dry eye disease, blepharitis and meibomian gland disease. Other disorders that can be treated include hyperemia of the eye and uveitis. The compositions can also be used for conjunctive epithelial healing such as from contact lens wear, corneal abrasion, or after a subject undergoes ocular surgery.

In accordance with the above, a pharmaceutical composition having a viscosity of 1 to 25 centipoise, for topical administration to the eye of a subject is disclosed, which includes mycophenolic acid or its pharmaceutically acceptable salt at a concentration of 0.05% to 0.3% wt/wt; and a glucosaminoglycan at a concentration of 0.1% to 2.5%. In some embodiments, a betamethasone or its pharmaceutically acceptable salt, such as betamethasone sodium phosphate, is also included at a concentration of 0.01% to 0.08% wt/wt.

In certain embodiments, the viscosity of the pharmaceutical composition is provided at 1 cp, 2 cp, 3 cp, 4 cp, 5 cp, 6 cp, 7 cp, 8 cp, 9 cp, 10 cp, 11 cp, 12 cp, 13 cp, 14 cp, 15 cp, 16 cp, 17 cp, 18 cp, 19 cp, 20 cp, 21 cp, 22 cp, 23 cp, 24 cp, or 25 cp. In certain embodiments, the viscosity is 1-5 cp, 1-10 cp, 1-20 cp, 10-20 cp, or 15-25 cp.

In certain embodiments the mycophenolic acid or pharmaceutically acceptable salt thereof, is provided at a concentration of, in % wt/wt, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30. In certain embodiments, the mycophenolic acid is selected from 0.05 to 0.10% wt/wt, 0.10 to 0.20% wt/wt, or 0.20 to 0.30% wt/wt.

In certain embodiments the pharmaceutical composition is steroid free. In other embodiments, the pharmaceutical composition includes betamethasone or a pharmaceutically acceptable salt thereof, at a concentration of, in % wt/wt, 0.01, 0.02, 0.03, 0.04, 0.05, 0.06, 0.07, or 0.08.

In certain embodiments, the glycosaminoglycan is provided at a concentration, in % wt/wt, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, or 2.5. Preferably the glycosaminoglycan is chondroitin sulfate at a concentration of 0.1% wt/wt to 2.5% wt/wt.

In certain embodiments the pharmaceutical compositions can also include a deturgescent agent and optionally other ingredients or excipients. A preferred deturgescent agent is a dextran or Dextran-70. Preferably, the dextran is provided at a concentration of 0.1% wt/wt to 5.0% wt/wt.

Accordingly, also disclosed is the use of the pharmaceutical composition having low viscosity for the treatment of an ocular disorder. Exemplary ocular disorders include dry eye disease, blepharitis, meibomian gland disease, conjunctival hyperemia, uveitis, and an ocular allergy. In some embodiments the pharmaceutical composition is for use after an ocular surface injury or after ocular surgery. In some embodiments, the pharmaceutical composition is used for the treatment of surgical temporary ocular discomfort (STODS). In some embodiments, the pharmaceutical composition is used for the treatment of corneal abrasion. In some embodiments, the pharmaceutical composition is used for the treatment of ocular discomfort after or during contact lens wear. Exemplary amounts of betamethasone or salt include 0.01% wt/wt to 0.08% wt/wt, 0.08% wt/wt to 0.1% wt/wt, or 0.1% wt/wt to 0.2% wt/wt. Exemplary amounts of mycophenolic acid or salt include 0.05% wt/wt to 0.30% wt/wt. A deturgescent agent could also be provided.

As such, a method of treating an ocular disorder is also provided, which includes administering to a subject suffering from an ocular disorder, a therapeutically effective amount of the pharmaceutical composition having low viscosity, wherein the pharmaceutical composition includes a primary active ingredient selected from the group consisting of betamethasone or a pharmaceutically acceptable salt thereof (e.g. betamethasone sodium phosphate), mycophenolic acid or a pharmaceutically acceptable salt there (e.g. mycophenolate sodium), and a combination of betamethasone and mycophenolic acid or a pharmaceutically acceptable salt(s) thereof in a pharmaceutically acceptable carrier. Preferably the pharmaceutical composition includes a glucosaminoglycan, most preferably chondroitin sulfate, at a concentration of 0.1% to 2.5% wt/wt. Among the ocular surface disorders that can be treated with the pharmaceutical compositions include dry eye disease, blepharitis, meibomian gland disease, conjunctival hyperemia, uveitis, and an ocular allergy. A subject's eye after ocular injury or after receiving an ocular surgery can also be treated using one or more of the pharmaceutical compositions. Nonlimiting examples include surgical temporary ocular discomfort (STODS), corneal abrasion and/or after or during contact lens wear. In preferred embodiments the pharmaceutical composition is topically administered as eye drops, optionally over weeks or months.

When the pharmaceutical composition includes betamethasone or the betamethasone salt (e.g. betamethasone sodium phosphate), it can be provided in a first preferred concentration of 0.01% wt/wt to 0.08% wt/wt. Such embodiments can be administered for only one, two, three or four weeks as a pulse approach. In an exemplary embodiment, the treatment is for an ocular disorder displaying conjunctival hyperemia. In such instances, conjunctival hyperemia can be reduced over extended periods such as over an additional 30, 60 or more than 60 days post administration when the primary active ingredient is betamethasone sodium phosphate administered for one, two, three or four weeks. Alternatively, betamethasone or its salt can be administered at 0.01% wt/wt to 0.08% wt/wt over many months without significant adverse events.

In other embodiments, the betamethasone or the betamethasone salt (e.g. betamethasone sodium phosphate) is provided at a concentration of 0.1%, 0.2% or about 0.2%. This embodiment is preferably only administered one to a few times per day for up to 14 days or two weeks.

In embodiments that include a primary ingredient including mycophenolic acid or a pharmaceutically acceptable salt thereof, preferably it is provided at a concentration of 0.05% wt/wt to 0.30% wt/wt. Such embodiments can be administered for only one, two, three or four weeks as a pulse approach. In an exemplary embodiment, the treatment is for an ocular disorder displaying conjunctival hyperemia. In such instances, conjunctival hyperemia can be reduced over extended periods such as over an additional 30, 60 or more than 60 days post administration when the primary active ingredient is mycophenolic acid administered for one, two, three or four weeks. Alternatively, mycophenolic acid or its salt can be administered at 0.05% wt/wt to 0.30% wt/wt over many months without significant adverse events.

In a related embodiment, a post-surgical ocular treatment method is also provided, which includes topically administering to an eye of a subject that has undergone ocular surgery, a therapeutically effective amount of the pharmaceutical composition having a low viscosity (e.g. 1-25 but preferably 1-10 centipoise), including a primary active ingredient selected from the group consisting of betamethasone or a pharmaceutically acceptable salt thereof (e.g. betamethasone sodium phosphate), mycophenolic acid or a pharmaceutically acceptable salt thereof (e.g. mycophenolate sodium), or a combination of both betamethasone and mycophenolic acid or pharmaceutically acceptable salts thereof. Each of the compositions preferably include a glucosaminoglycan, such as chondroitin sulfate, at a concentration of 0.1% to 2.5% wt/wt. Preferred compositions include 0.01% wt/wt to 0.08% wt/wt betamethasone sodium phosphate, 0.05% wt/wt to 0.30% wt/wt mycophenolic acid or both 0.01% wt/wt to 0.08% wt/wt betamethasone sodium phosphate and 0.05% wt/wt to 0.30% wt/wt mycophenolic acid. Such treatments can be administered regularly (e.g. one to three times daily) over periods of between one week and many months, such as one week, two weeks, three weeks, four weeks or one month, two months, three months, or more, without adverse effects associate with higher concentration steroid-based therapeutic approaches. In instances where severe inflammation occurs, an additional pharmaceutical composition containing 0.2% wt/wt betamethasone sodium phosphate may be preferred for short term treatment (eg. only 1 to 2 weeks maximum with minimal dosing per day).

In still another related embodiment, a method of treating hyperemia of the eye is provided, which includes administering to the eye of a subject suffering from hyperemia, a therapeutically effective amount of the pharmaceutical composition having a low viscosity, including a primary active ingredient selected from the group consisting of mycophenolic acid or a pharmaceutically acceptable salt thereof (e.g. mycophenolate sodium), betamethasone or a pharmaceutically acceptable salt thereof (e.g. betamethasone sodium phosphate), or a combination of both mycophenolic acid and betamethasone or pharmaceutically acceptable salt(s) thereof. In some embodiments the pharmaceutical composition includes mycophenolic acid or salt at a concentration from 0.05% to 0.30% wt/wt or betamethasone sodium phosphate at a concentration of 0.01% to 0.08% wt/wt. Each of the compositions preferably include a glucosaminoglycan, such as chondroitin sulfate, at a concentration of 0.1% to 2.5% wt/wt In some embodiments, the composition includes 0.05% to 0.30% wt/wt mycophenolic acid and 0.01% to 0.08% betamethasone sodium phosphate. The treatment can be administered regularly (e.g. one to three times daily) over periods of between one week and many months, such as one week, two weeks, three weeks, four weeks or one month, two months, three months, or more.

In another related embodiment, a method of treating uveitis is provided, which includes administering to an eye of a subject suffering from uveitis, a therapeutically effective amount of the pharmaceutical composition having low viscosity, including a primary active ingredient selected from the group consisting of mycophenolic acid or a pharmaceutically acceptable salt thereof (e.g. mycophenolate sodium), betamethasone or a pharmaceutically acceptable salt thereof (e.g. betamethasone sodium phosphate), or mycophenolic acid and betamethasone or pharmaceutically acceptable salts thereof. In some embodiments the composition includes mycophenolic acid or salt at a concentration from 0.05% to 0.30% wt/wt or betamethasone or salt at a concentration of 0.01% to 0.08% wt/wt. In some embodiments, the composition includes f 0.05% to 0.30% wt/wt mycophenolic acid or salt and 0.01% to 0.08% betamethasone or salt. In each embodiment, the composition also includes a glycosaminoglycan, preferably chondroitin sulfate, at 0.1% to 2.5% wt/wt. The treatment can be administered regularly (e.g. one to three times daily) over periods of between one week and many months, such as one week, two weeks, three weeks, four weeks or one month, two months, three months, or more.

In another related embodiment, a method of conjunctive epithelial cell healing is provided, which includes administering to an eye of a subject suffering from damage to conjunctive epithelial cells, a therapeutically effective amount of the pharmaceutical composition having low viscosity, including a primary ingredient selected from the group consisting of mycophenolic acid or a pharmaceutically acceptable salt thereof (e.g. mycophenolate sodium), betamethasone or a pharmaceutically acceptable salt thereof (e.g. betamethasone sodium phosphate), or both mycophenolic acid and betamethasone or their pharmaceutically acceptable salts. In some embodiments the composition includes mycophenolic acid or salt at a concentration from 0.05% to 0.30% wt/wt or betamethasone or salt at a concentration of 0.01% to 0.08% wt/wt. In some embodiments, the composition includes 0.01% to 0.3% wt/wt mycophenolic acid or salt and 0.01% to 0.08% betamethasone or salt. In each embodiment, the composition also includes a glycosaminoglycan, such as chondroitin sulfate, preferably at 0.1% to 2.5% wt/wt. In some embodiments the epithelial cells in need of healing are damaged from an ocular surgery. The treatment can be administered regularly (e.g. one to three times daily) over periods of between one week and many months, such as one week, two weeks, three weeks, four weeks or one month, two months, three months, or more.

### BRIEF DESCRIPTION OF THE DRAWINGS

FIG. 1 is graph summarizing results from a study assessing the decrease in conjunctive hyperemia in subjects over time after treatment with a low viscosity composition containing mycophenolic acid (MPA) administered in the form of mycophenolate sodium, betamethasone sodium phosphate (BSP), or a combination of MPA and BSP.

FIG. 2 is graph summarizing results from a study assessing the decrease of University of North Carolina Dry Eye Management Scale (UNC DEMS) value over time between treatment with a low viscosity composition containing MPA administered in the form of mycophenolate sodium, betamethasone sodium phosphate (BSP), and a combination of MPA and BSP.

FIG. 3 is graph summarizing results from a study assessing the decrease in hyperemia in subjects over time after treatment with low viscosity 0.3% MPA/0.1% BSP (SURF-100); 5% lifitegrast (XIIDRA); 0.05% cyclosporine (RESTASIS); low viscosity 0.1% MPA; low viscosity 0.3% MPA; and vehicle control for SURF-100 and MPA solutions.

FIG. 4 is graph comparing UNC DEMS values over approximately 70 contiguous days from subjects treated with a low viscosity 0.02% BSP or 0.04% BSP over a two-week pulsed dosing period.

FIG. 5 is a graph comparing the absolute change from baseline in conjunctival hyperemia over approximately 70 contiguous days from subjects treated with a low viscosity 0.02% BSP or 0.04% BSP over a two-week pulsed dosing period.

FIG. 6 is a graph comparing the absolute change from baseline in anesthetized Schirmer testing over approximately 70 contiguous days from subjects treated with a low viscosity 0.02% BSP or 0.04% BSP over a two-week pulsed dosing period.

FIG. 7 is a graph comparing the absolute change from baseline in tear break-up time (TBUT) analysis over approximately 70 contiguous days from subjects treated with a low viscosity 0.02% BSP or 0.04% BSP over a two-week pulsed dosing period.

### DETAILED DESCRIPTION OF PREFERRED EMBODIMENTS

The subject matter described herein may be embodied in different forms and should not be construed as limited to the following exemplary embodiments. Indeed, many modifications and other embodiments of the presently disclosed subject matter will come to mind to one having ordinary skill in the art to which the presently disclosed subject matter pertains having the benefit of the teachings presented in the descriptions included herein. Therefore, it is to be understood that the presently disclosed subject matter is not to be limited to the specific embodiments disclosed and that modifications and other embodiments are intended to be included within the scope of the disclosed subject matter.

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

The term “about” as used herein means that the number referred to includes the recited number plus or minus 1-10% of that recited number. For example, an ingredient making up “about” 90% of a solution could be 85%, 86%, 87%, 88%, 89% 90%, 91% 92%, 93%, 94%, 95% depending on the amount of remaining ingredients. A pH of “about” 7.3 could be 7.1, 7.2, 7.3, 7.4, or 7.5. Whenever it appears herein, a numerical range such as “1 to 20” refers to each integer in the given range; i.e., meaning only 1, only 2, only 3, etc., up to and including only 20.

The term “active ingredient” refers to any component that provides pharmacological activity or direct effect in the diagnosis, cure, mitigation, treatment, or prevention of disease or to affect the structure or any function of the body of mammals, namely, humans. In contrast, inactive ingredients are usually considered excipients. A “primary active ingredient” as used herein refers to a first active ingredient when more than one active ingredient may be present in a composition.

The terms “administration of a pharmaceutical composition” or “administering a pharmaceutical composition” are defined to include an act of providing a compound or pharmaceutical composition of the invention to a subject in need of treatment. A “pulsed” administration approach refers to a shortened period, such as administration of one week, two weeks, three weeks or four weeks. The term “administered up to four weeks” means administration is performed over a period of up to four weeks, such as one week, two weeks, three weeks or four weeks. Unless otherwise specified, the preferred route of administration is topical application to the eye of the subject. When provided as eye drops, the “administration of a pharmaceutical composition” is preferably a single drop per eye but can be two or three drops per eye. The term “subject” means a mammal and is preferably a human.

The term “albumin” refers to any non-glycosylated proteins found in blood plasma.

The term “betamethasone or a pharmaceutically acceptable salt thereof at a concentration of 0.01% wt/wt to 0.08% wt/wt” means that when the betamethasone is provided in the form of betamethasone sodium phosphate, the concentration of betamethasone sodium phosphate would be from 0.01% wt/wt to 0.08% wt/wt. Since betamethasone has an approximately 25% lower molecular weight than betamethasone sodium phosphate (the molecular weight of betamethasone is 392 compared to 516 for betamethasone sodium phosphate), these ranges can differ up to 25% or about 25% when using betamethasone alone to achieve a same concentration of betamethasone.

The terms “consisting essentially of” and “consists essentially of” refer to inclusion of the recited ingredients or components without additional components or ingredients that would materially alter the therapeutic effect. The term “consisting essentially of” is not intended to exclude excipients, such as pharmaceutical carriers, lubricants, stabilizers, flavorings, deturgescent agents and the like unless expressly mentioned. The term “consisting essentially of” and “consists essentially of” may be replaced with “containing as the only active ingredient(s)” or “the only ingredients effecting the underlying disease or condition.”

The term “contiguous” refers to a series of one immediately after another without interruption. The term “period of 70 contiguous days” refers to a time period that extends 1,680 hours (70 days×24 hours). A reduction in symptoms for a “period of 70 contiguous days” means that on a first day and 70 days later the symptom is reduced. The symptom is preferably reduced for each of the contiguous days, but this is not required.

The term “corticosteroid” refers to any steroid hormone, both produced synthetically and obtained from the adrenal cortex of vertebrates (inclusive of both glucocorticoids and mineralocorticoids) and belonging to a sub-genus of steroids that are derivatives of corticosterone. The term “day” refers to a 24 hour time period.

The term “deturgescent agent” refers to a compound that is capable of maintaining the stroma of the cornea of the eye in a state of relative dehydration to an extent necessary to ensure the transparency of the cornea. A “deturgescent agent” can affect corneal deturgescence.

The term “glycosaminoglycan” refers to any unbranched polysaccharide having a repeating disaccharide unit. The preferred glycosaminoglycan for use in the methods and compositions described throughout this disclosure is chondroitin sulfate. Other glycosaminoglycans include dermatan sulfate, dermatin sulfate, heparan sulfate, keratin sulfate, keratan sulfate, and hyaluronic acid. Since glycosaminoglycans are known to increase viscosity of formulations, their use herein is used under tight tolerances to maintain a pharmaceutical composition with a “low viscosity”.

The term “low viscosity” refers to a formulation having a viscosity about the same as water to permit facile administration to the ocular surface without substantial resistance to flow, such as by instillation as eye drops. In particular, the low viscosity formulation has a viscosity from about 1 centipoise (cp) to about 25 cp, including any subranges or values therein (e.g., about 1-20 cp, about 1-15 cp, but preferably 1-10 cp), as measured under standard conditions (e.g., at approximately 20-25° C. using a conventional viscometer). This “low viscosity” is maintained notwithstanding the presence of viscosity-modifying agents. For example, although components such as chondroitin sulfate and dextran may contribute to increased viscosity, the formulation is adjusted (e.g., via concentration selection or inclusion of additional excipients) such that the overall viscosity remains within the low viscosity range defined herein. The “low viscosity” formulations, as described herein, are distinguishable from more viscous ophthalmic compositions (e.g., gels, ointments, or highly thickened solutions) and provides improved spreadability across the ocular surface while minimizing blurring and discomfort associated with higher viscosity systems.

The term “minimal dosing per day” refers to administration of preferably one drop per eye per day but may be two or three drops. The skilled artisan will appreciate that the volume of an eye drop may vary depending on the formulation but is generally about 40 μL-50 μL.

The term “mycophenolic acid” or “MPA” refers to the compound having the IUPAC name 6-(4-hydroxy-6-methoxy-7-methyl-3-oxo-1H-2-benzofuran-5-yl)-4-methylhex-4-enoic acid and the following chemical structure:

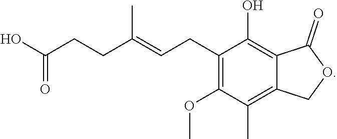 [SMILES: COc1c(C)c2c(c(O)c1C/C=C(\C)CCC(=O)O)C(=O)OC2]

or a pharmaceutically acceptable salt of mycophenolic acid, including but not limited to mycophenolate or mycophenolate sodium.

The term “mycophenolic acid or a pharmaceutically acceptable salt thereof at a concentration of 0.05% wt/wt to 0.30% wt/wt” means that the concentration of mycophenolic acid in the composition is from 0.05% wt/wt to 0.30% wt/wt. Therefore, when mycophenolic acid is provided in the form of mycophenolate or mycophenolate sodium, the amount of mycophenolate or mycophenolate sodium may be higher due to the higher molecular weight to achieve mycophenolic acid at 0.05% wt/wt to 0.30% wt/wt. The molecular weight of mycophenolate or mycophenolate sodium is about 7% higher than mycophenolic acid and would therefore be provided in amount that is about 7% higher to achieve an equal amount of mycophenolic acid.

The term “ocular disorder” refers to a medical condition related to the eye and includes “ocular surface disease” (including “dry eye”, “dry eye syndrome” and “dry eye disease”), which is defined as one or several conditions associated with, or caused by, either decreased or insufficient tear production or increased or excessive tear film evaporation, or both, and characterized by redness, itching, and/or burning of the eye. An ocular surface disease is further defined as being inclusive of keratoconjunctivitis sicca, episodic dry eye disease, chronic dry eye disease, recalcitrant dry eye disease, preservative induced ocular surface disease, contact lens induced ocular surface dry eye disease, age-related dry eye, neurotrophic ocular surface disease, meibomian gland disease (MGD) and blepharitis. Ocular surface diseases such as at least some of those listed above also commonly occur following ocular surgery procedures, such as cataract surgery or refractive surgery. Ocular disorders also include ocular pain or discomfort from ocular allergy, a corneal abrasion, contact lens wear, STODS, ocular surgery, and an ocular surface disease.

As used herein, “or” means “and/or” unless stated otherwise. Furthermore, use of the term “including” as well as other forms, such as “includes,” and “included,” is not limiting.

The term “pharmaceutical composition” is defined as a chemical or a biological compound or substance, or a mixture or combination of two or more such compounds or substances, intended for use in the medical diagnosis, cure, treatment, or prevention of disease or pathology. A “pharmaceutical composition” includes an active ingredient provided in a pharmaceutically acceptable carrier.

The term “pharmaceutically acceptable,” when used in the context of a carrier, is defined as a carrier, whether diluent or excipient, that is compatible with the other ingredients of the formulation and not deleterious to the recipient thereof.

The term “salt” refers to an ionic compound which is a product of the neutralization reaction of an acid and a base. The term “salt” when used in a pharmaceutical is a pharmaceutically acceptable salt. For example, mycophenolate sodium is a pharmaceutically acceptable salt of mycophenolic acid and betamethasone sodium phosphate is a pharmaceutically acceptable salt of betamethasone.

The terms “solvate” and “hydrate” are used herein to indicate that a compound or a substance is physically or chemically associated with a solvent for “solvates” such as water (for “hydrates”).

The term “therapeutically effective amount” is defined as the amount of the compound or pharmaceutical composition that will elicit the biological or medical response of a tissue, system, animal or human that is being sought by the researcher, medical doctor or other clinician.

The terms “treat”, “treating”, and “treatment” refer to the reduction or amelioration of one or more signs or symptoms of an ocular disorder or disease. Medically, the term “symptom” refers to feelings or sensations that a subject may have, and the term “sign” refers to observable characteristics. Where “symptom” or “sign” is disclosed herein, one or both is intended to be encompassed. Examples of signs and symptoms can vary depending on the particular ocular disorder or disease being treated but may include hyperemia or redness, itching, burning of the eye, eye discomfort and eyelid discomfort.

### B. Pharmaceutical Compositions

This disclosure documents pharmaceutical compositions having low viscosity and improved efficacy for the treatment of ocular disorders, which contain mycophenolic acid or a pharmaceutically acceptable salt thereof (e.g. mycophenolate sodium) and/or betamethasone or a pharmaceutically acceptable salt thereof (e.g. betamethasone sodium phosphate), most preferably with a glucosaminoglycan.

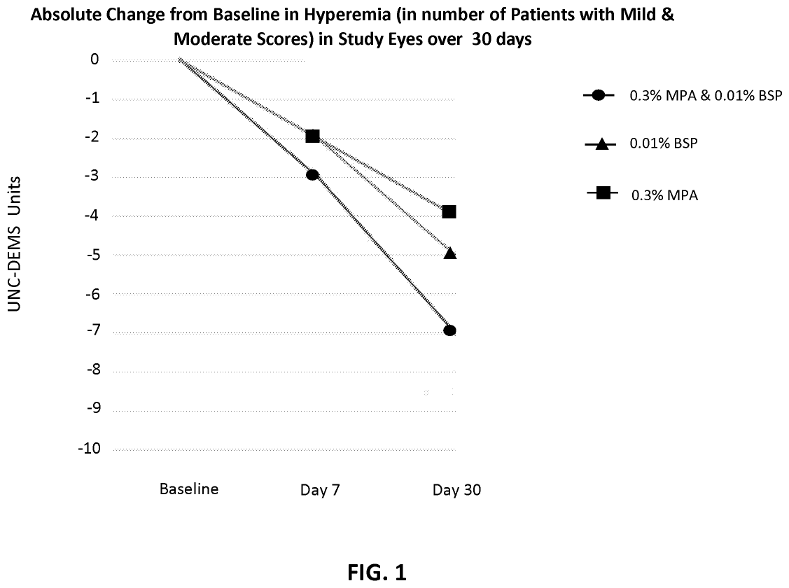

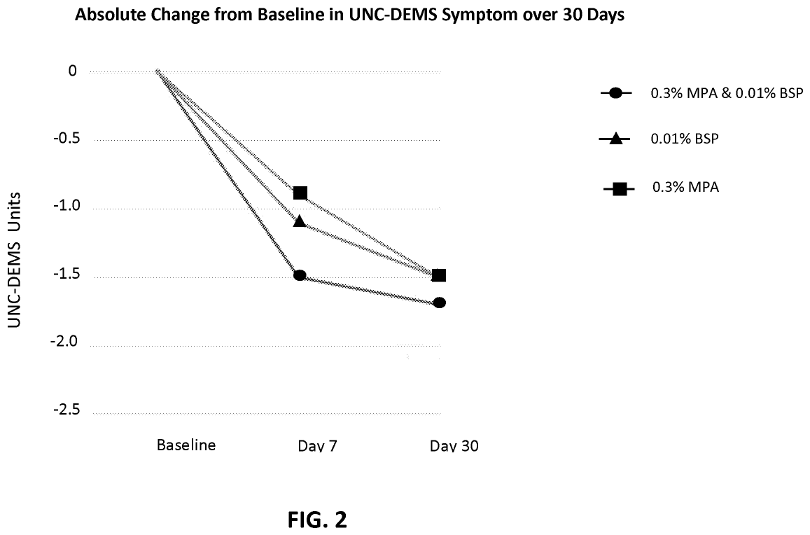

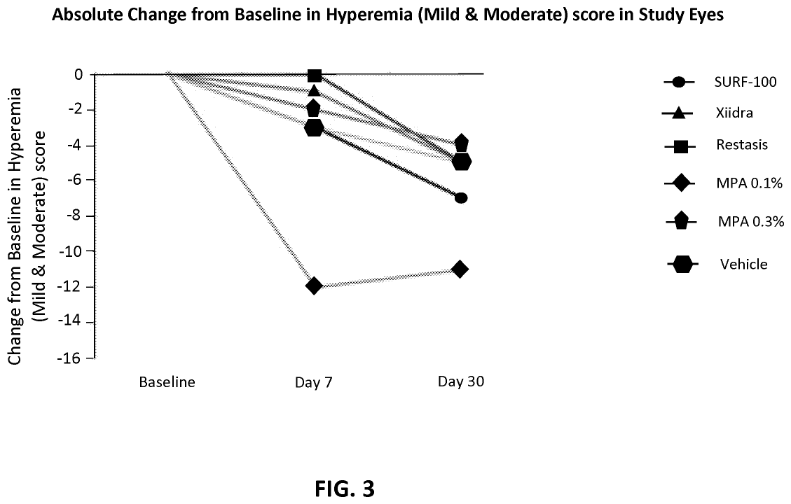

Among the improvements disclosed herein include the synergistic effects in the treatment of ocular disorders, surprisingly discovered when combining a low dose of mycophenolic acid and a low dose of the glycosaminoglycan chondroitin sulfate in an ocular formulation at low viscosity. Still further surprising benefits were found when adding betamethasone sodium phosphate also at low dose. An example of these synergistic effects can be seen for example in FIG. 1, where a significant reduction in hyperemia of the eye was regularly observed in subjects suffering from an ocular surface disease when a combination of a low viscosity 0.3% mycophenolic acid (MPA) and 0.01% betamethasone sodium phosphate (BSP) was topically applied compared to 0.3% MPA and 0.01% BPA administered without the other. As further confirmation of these synergistic effects, FIG. 2 demonstrates a significant reduction in scoring using the University of North Carolina Dry Eye Management Scale (UNC-DEMS) in subjects suffering from an ocular surface disease when a combination of a low viscosity 0.3% MPA and 0.01% BSP was topically applied compared to 0.3% MPA and 0.01% BPA without the other. As shown in FIG. 2, subjects rated their treatment with a low viscosity 0.3% MPA or 0.01% BSP individually as improved but about the same as one another. However significant improvement was scored in the combined treatment. This was even further surprising looking at the results of FIG. 3, where a low viscosity 0.1% MPA worked much better than 0.3% MPA, which demonstrates the still lower dose of MPA is more efficacious than the higher, yet still low dose. Still further, this synergistic combination of MPA and BSP was compared to conventional treatments XIIDRA and RESTASIS. FIG. 3 demonstrates a significant reduction in hyperemia in subjects suffering from an ocular surface disease when a combination of low viscosity 0.3% MPA, 0.01% BSP (SURF-100) was topically applied compared to XIIDRA and RESTASIS. Also shown in FIG. 3 is the reduction in hyperemia when topically administering low viscosity 0.3% MPA and the unexpectedly high reduction in hyperemia when topically administering 0.1% MPA. Moreover, the subjects tested did not report adverse events associated with XIIDRA and RESTASIS when using the MPA, BSP or MPA/BSP formulations.

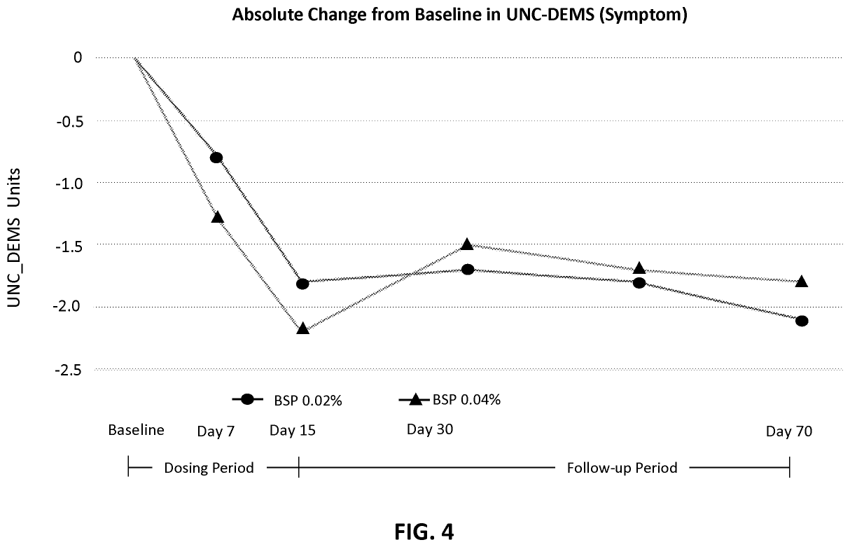

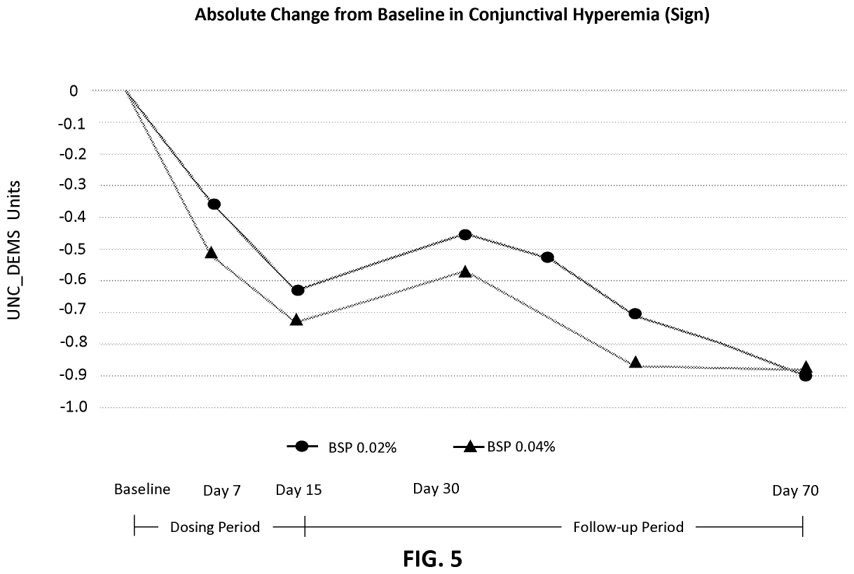

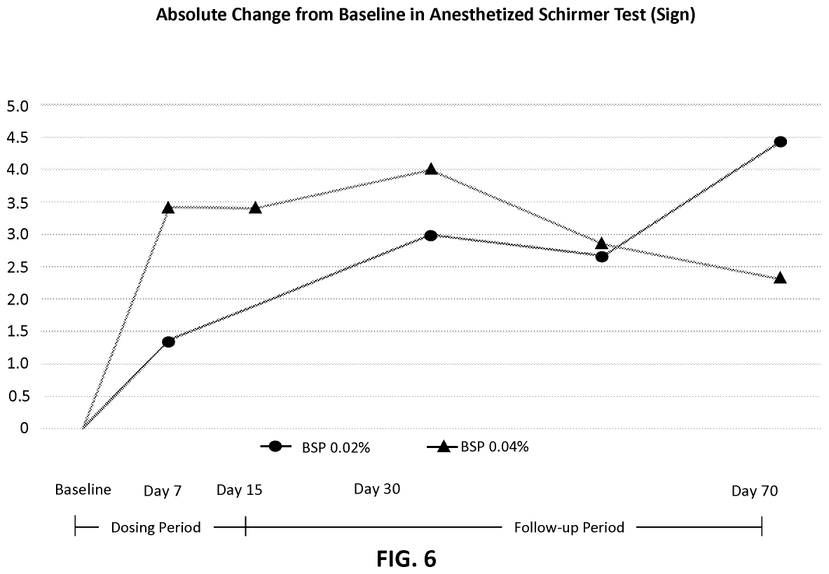

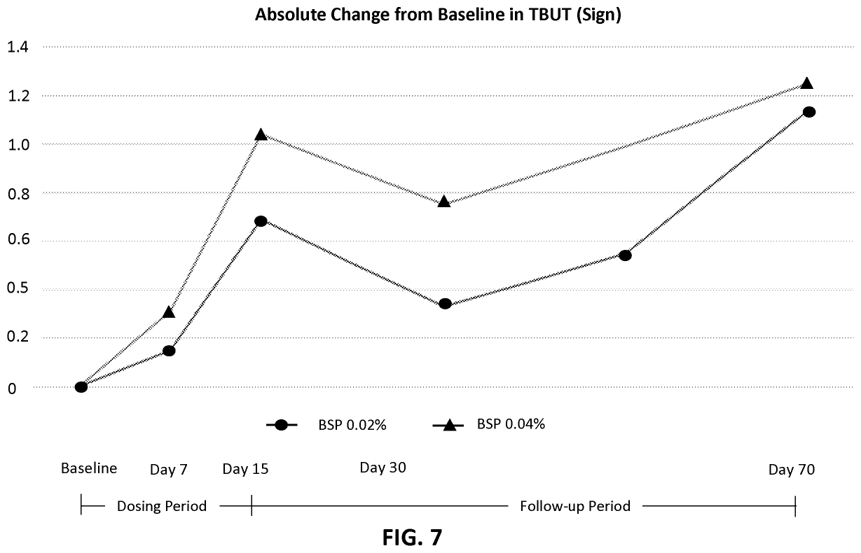

In a different study, potential extended benefits after only administering low viscosity betamethasone sodium phosphate (BSP) for a shortened time and at low dose were also studied. The testing is described in more detail in Example 8 but broadly, subjects suffering from an ocular disorder with symptoms and signs of eye redness, eye discomfort, and poor tear film quality were given either low viscosity 0.02% BSP or 0.04% BSP twice daily for two weeks only. In summary, FIG. 4 shows that eye discomfort was significantly relieved for the entire testing period (70 days) in both groups. FIG. 5 shows that hyperemia was significantly reduced over the entire testing period in both groups. FIG. 6 and FIG. 7 confirm that tear film was improved over the entire treatment period in both groups. Further, adverse events associated with administration of corticosteroids generally were not reported.

The surprising effect of providing the low dose of MPA and/or BSP, is believed to be at least in part because the low viscosity formulations also included a low dose of chondroitin sulfate. Glycosaminoglycans such as chondroitin sulfate are conventionally employed in ophthalmic formulations at relatively high concentrations to increase viscosity and provide a protective, lubricating coating over the ocular surface, for example, in viscoelastic compositions used prior to or during ocular surgery. Such formulations are typically designed to form a thickened, persistent layer and are not optimized for low-viscosity delivery. In contrast, it has been surprisingly found that inclusion of chondroitin sulfate at relatively low concentrations, for example from about 0.1% to about 2.5% (wt/wt), in a low-viscosity formulation (e.g., about 1-25 cp but preferably 1-10 cp) provides a surprising therapeutic benefit without imparting from the high viscosity traditionally associated with glycosaminoglycan-containing compositions. Without being bound by theory, it is believed that even at these low concentrations, chondroitin sulfate may protect epithelial tissues, thereby supplementing the therapeutic efficacy of the formulation. Notably, these benefits are achieved while maintaining a viscosity close to that of water, preserving desirable administration characteristics such as rapid spreading, minimal visual disturbance, and improved patient comfort. This result is unexpected in view of the conventional understanding that glycosaminoglycans function primarily through formation of viscous coatings at higher concentrations.

Accordingly, the low viscosity pharmaceutical compositions described herein are effective even at very low concentrations, such as betamethasone sodium phosphate at 0.01% wt/wt and mycophenolic acid at 0.05% wt/wt. Among the significant improvements over the art are that by significantly decreasing the concentration of a steroid component compared to currently prescribed steroid-based formulations, adverse effects such as increased intraocular pressure, which is a glaucoma risk factor, cataract, infection, and wound-healing delay are eliminated or at least significantly reduced. To this end, the disclosed compositions are suitable for administration for extended periods of time to significantly relieve redness, itching, and burning of the eyes in subjects suffering from ocular disorders presenting such signs and symptoms, such as but not limited to dry eye disease, blepharitis, meibomian gland disease, ocular allergy.

The disclosed low viscosity pharmaceutical compositions are useful for treating, preventing, and/or alleviating signs and symptoms of ocular disorders, such as ocular surface disease (e.g. dry eye disease, blepharitis, meibomian gland disease, ocular allergy). In various embodiments, the compositions include 0.01% wt/wt to 0.08% wt/wt betamethasone or a pharmaceutically acceptable salt thereof; and/or 0.05% to 0.30% wt/wt mycophenolic acid or a pharmaceutically acceptable salt thereof. They also contain a glycosaminoglycan, such as chondroitin sulfate, at 0.1% to 2.5% wt/wt. In some embodiments these are the sole active ingredients but in other embodiments, one or more additional ingredient is provided.

Betamethasone and pharmaceutical acceptable salts of betamethasone, such as betamethasone sodium phosphate, are in a class of medications called corticosteroids. Corticosteroids are a type of anti-inflammatory drug. They are generally known to provide relief for inflamed areas of the body. They lessen swelling, redness, itching and allergic reaction. Corticosteroids are prescribed as a treatment for several different diseases. EYEBET, which is not currently available in the US, provides a 0.1% solution of betamethasone sodium phosphate for the short-term treatment of inflammation. Among the potential side effects of EYEBET include irritation, burning, stinging, and itching of the eyes. Blurred or clouded vision may also occur. Moreover, prolonged use of 0.1% betamethasone sodium phosphate may lead to formation of posterior subcapsular cataracts. Within the present invention betamethasone and its pharmaceutically acceptable salts can be used at a significantly lower concentration than previously suggested, such as from 0.01% to 0.08% wt/wt (e.g. 0.01%, 0.02%, 0.03%, 0.04%, 0.05%, 0.06%, 0.07%, 0.08% wt/wt or amounts therebetween) when used topically in the treatment of ocular disorders, such as dry eye disease, or after undergoing ocular surgery. These concentration guidelines are based on amounts of betamethasone sodium phosphate. One having ordinary skill in the art to which the invention belongs will appreciate that the concentration of the betamethasone compound could differ by as much as about 25% in the formulations since betamethasone has a molecular weight that is about 25% less than betamethasone sodium phosphate (betamethasone sodium phosphate has a molecular weight of 516 compared to a molecular weight of 392 for betamethasone). Thus, betamethasone would be added in an amount that is about 25% less compared to betamethasone sodium phosphate. Whether betamethasone sodium phosphate or betamethasone, this lower concentration significantly reduces the adverse effects of steroids commonly found at higher dosages. This was confirmed when testing the effect of administration of low doses of betamethasone over longer periods of time. In particular, betamethasone sodium phosphate was administered at low doses without subjects reporting adverse events (see Example 7; FIGS. 1-2). Subsequent testing also showed that pulsed administration of even low doses of betamethasone sodium phosphate (e.g. two weeks) can result in relief of signs and symptoms of ocular disorders for months afterwards without adverse events (see Example 8; FIGS. 4-7). Though these low doses of betamethasone worked surprisingly well, higher doses such as 0.09% to 0.1% may also be used, and dosages of 0.1% to 0.2% can also be used sparingly such as over two weeks or fewer.

Mycophenolic acid is an immunosuppressant, and thus lowers the activity of the immune system. In particular, it prevents the proliferation of T-cells, lymphocytes and the formation of antibodies from B-cells. It is most commonly known for its use in organ transplantation, such as after kidney, heart and liver transplantation. It is also used to treat autoimmune conditions such as Crohn's disease and lupus. Mycophenolic acid has also been proposed for use with inflammatory eye diseases; however, these studies relied on oral treatment rather than topical applications to the eye. For example, Willeke (2017), conducted a study of 11 subjects having primary Sjögren syndrome, where orally administered mycophenolate sodium (MPS) resulted in in a subjective improvement of ocular dryness on a visual analogue scale and a reduced demand for artificial tear supplementation.

It has been surprisingly found that administration of mycophenolic acid at very low concentration can significantly reduce certain signs and symptoms associated with ocular surface disease. Surprisingly, FIG. 3 shows that 0.1% wt/wt mycophenolic acid was even more effective than 0.3% wt/wt. Thus, in some embodiments, a composition is provided which includes mycophenolic acid or its pharmaceutically acceptable salt and a glycosaminoglycan (e.g. chondroitin sulfate). Exemplary amounts of mycophenolic acid are from 0.01% wt/wt to 0.3% wt/wt. In some embodiments the dosage of mycophenolic acid or its pharmaceutically acceptable salt is selected from the group consisting of 0.01% wt/wt, 0.02% wt/wt, 0.03% wt/wt, 0.4% wt/wt, 0.05% wt/wt, 0.06% wt/wt, 0.07% wt/wt, 0.8% wt/wt, 0.9% wt/wt, 0.10% wt/wt, 0.11% wt/wt, 0.12% wt/wt, 0.13% wt/wt 0.14% wt/wt, 0.15% wt/wt, 0.16% wt/wt, 0.17% wt/wt, 0.18% wt/wt, 0.19% wt/wt, 0.20% wt/wt, 0.21% wt/wt, 0.22% wt/wt, 0.23% wt/wt, 0.24% wt/wt, 0.25% wt/wt, 0.26% wt/wt, 0.27% wt/wt, 0.28% wt/wt, 0.29% wt/wt, 0.30% wt/wt, and amounts therebetween in a low viscosity pharmaceutical composition formulated as eye drops for topical use on the eye. These concentration guidelines are based on amounts of mycophenolic acid.

When using a salt such as mycophenolate or mycophenolate sodium, the amounts can be adjusted so that the mycophenolic acid is present at the described ranges.

As shown in FIGS. 1-3, combining mycophenolic acid together with the substantially decreased dosage of betamethasone sodium phosphate and together with low dose of chondroitin sulfate, provides an effective and synergistic treatment for ocular surface disease, such as dry eye disease, when applied topically to the eye of subjects. In particular, when combining betamethasone sodium phosphate at a concentration from 0.01. % to 0.08% wt/wt (e.g. 0.01% wt/wt, 0.02% wt/wt, 0.03% wt/wt, 0.04% wt/wt, 0.05% wt/wt, 0.06% wt/wt, 0.07% wt/wt, 0.08% wt/wt, or amounts therebetween), mycophenolic acid at a concentration from 0.01% to 0.30% wt/wt (e.g. 0.01% wt/wt, 0.02% wt/wt, 0.03% wt/wt, 0.4% wt/wt, 0.05% wt/wt, 0.06% wt/wt, 0.07% wt/wt, 0.8% wt/wt, 0.9% wt/wt, 0.10% wt/wt, 0.11% wt/wt, 0.12% wt/wt, 0.13% wt/wt 0.14% wt/wt, 0.15% wt/wt, 0.16% wt/wt, 0.17% wt/wt, 0.18% wt/wt, 0.19% wt/wt, 0.20% wt/wt, 0.21% wt/wt, 0.22% wt/wt, 0.23% wt/wt, 0.24% wt/wt, 0.25% wt/wt, 0.26% wt/wt, 0.27% wt/wt, 0.28% wt/wt, 0.29% wt/wt, 0.30% wt/wt, or amounts therebetween) and a glucsaminoglycan, such as chondroitin sulfate, at a concentration of 0.1% to 2.5% wt/wt (e.g. 0.10% wt/wt, 0.11% wt/wt, 0.12% wt/wt, 0.13% wt/wt 0.14% wt/wt, 0.15% wt/wt, 0.16% wt/wt, 0.17% wt/wt, 0.18% wt/wt, 0.19% wt/wt, 0.20% wt/wt, 0.21% wt/wt, 0.22% wt/wt, 0.23% wt/wt, 0.24% wt/wt, 0.25% wt/wt, or amounts therebetween) in a low viscosity pharmaceutical composition formulated as eye drops for topical use on the eye, an effective treatment of signs and symptoms for ocular surface disease, such as dry eye disease, is achieved. As such, this combination is also useful as a treatment for blepharitis, meibomian gland disease, conjunctival hyperemia, uveitis, ocular allergy and as a treatment after undergoing ocular surgery, where the subject is at risk of developing such conditions. In addition to its beneficial effects, this combination eliminates or at least significantly reduces unwanted treatment effects found with off label use (e.g. over days to weeks) of higher concentrations of compounds like 0.1% betamethasone sodium phosphate.

The pharmaceutical compositions of betamethasone and/or mycophenolic acid or their pharmaceutically acceptable salts maintain a low viscosity despite the presence of at least one glycosaminoglycan, which is conventionally used to increase viscosity. It can be theorized, without firm commitment to any particular or specific mechanism, that at low dose (e.g. 1-2.5% wt/wt), glycosaminoglycans may be useful in protecting endothelial and epithelial cells which are subject to exposure to trauma, and/or to promote the growth of such cells. Non-limiting examples of glycosaminoglycan(s) that may be used include: chondroitin, chondroitin sulfate, dermatan sulfate, dermatin sulfate, heparin sulfate, heparan sulfate, keratin sulfate, keratan sulfate, or hyaluronic acid. The preferred glycosaminoglycan is chondroitin sulfate. The total contents of the glycosaminoglycan(s) in the composition expressed as the mass concentration may be from 0.1% wt/wt to 5.0% wt/wt, such as from 0.2% wt/wt to 4.0% wt/wt, for example 0.1% wt/wt, 0.15% wt/wt. 0.2% wt/wt, 0.25% wt/wt, 0.3% wt/wt, 0.35% wt/wt, 0.4% wt/wt, or amounts therebetween. Preferably, the concentration is 0.1% to 2.5% wt/wt.

The pharmaceutical compositions can also include at least one deturgescent agent. The preferred deturgescent agent is dextran, namely, Dextran-70. However, non-limiting examples of other acceptable deturgescent agent(s) that may be used in addition to, or instead of, dextran include any of: dextran sulfate, NaCl, dextrose, and sucrose. While such deturgescent agents are typically used to provide dehydration for stroma of the cornea of the eye, as defined above, unexpectedly, deturgescent agents used in the compositions disclosed herein are also beneficial for improving outcomes in the process of treatment of various surface ocular diseases such as dry eye disease and after ocular surgery despite their low concentration. The total contents of the deturgescent agent(s) in the composition expressed as the mass concentration may be from 0.1% wt/wt to 5.0% wt/wt, such as from 0.2% wt/wt to 4.0% wt/wt, for example 0.25% wt/wt.

In some embodiments, any one or more of the compositions may also include one or more antioxidants such as ascorbic acid or ascorbic acid derivatives, erythorbic acid, and sodium ascorbate; Thiol derivatives such as thioglycerol, cysteine, acetylcysteine, cystine, dithioerythreitol, dithiothreitol, glutathione; Tocopherols; butylated hydroxyanisol (BHA); butylated hydroxytoluene (BHT); sulfurous acid salts such as sodium sulfate, sodium bisulfite, acetone sodium bisulfite, sodium metabisulfite, sodium sulfite, sodium formaldehyde sulfoxylate, and sodium thiosulfate; and nordihydrogualaretic acid.

In related embodiments of the present invention, pharmaceutical compositions having fewer than the above-recited components are provided for treating, preventing, and/or alleviating an ocular surface disease. In various embodiments, the compositions may comprise or consist essentially of betamethasone or a pharmaceutically acceptable salt thereof at 0.01% to 0.08% wt/wt or mycophenolic acid or a pharmaceutically acceptable salt thereof at 0.05% wt/wt to 0.30% wt/wt; and a pharmaceutically acceptable carrier that is suited for topical administration to the eye. Preferably, the viscosity is about the same as water and includes 0.1% to 2.5% glycosaminoglycan (e.g. chondroitin sulfate). While in some embodiments, the betamethasone or salt, and/or mycophenolic acid or salts is provided as a sole active ingredient, in other embodiments, the composition may include one or more of: tacrolimus, cyclosporine, albumin, plasma, platelet-rich plasma, serum, and pharmaceutically acceptable salts, hydrates, solvates, esters thereof or derivatives or analogs thereof.

In those embodiments where the compositions comprise, consist of, or consist essentially of betamethasone and/or mycophenolic acid or their pharmaceutically acceptable salt(s) thereof, the formulation can be present in a solution either as a part of a polycarbophil-based formulation or as a part of a non-polycarbophil-based formulation as long as the viscosity is from about 1 cp to about 25 cp, preferably 1-10 cp. In various embodiments, the total content of the mycophenolic acid in the composition expressed as the mass concentration may be from 0.01% wt/wt to 0.3% wt/wt, such as 0.01% wt/wt, 0.02% wt/wt, 0.03% wt/wt, 0.4% wt/wt, 0.05% wt/wt, 0.06% wt/wt, 0.07% wt/wt, 0.8% wt/wt, 0.9% wt/wt, 0.10% wt/wt, 0.11% wt/wt, 0.12% wt/wt, 0.13% wt/wt 0.14% wt/wt, 0.15% wt/wt, 0.16% wt/wt, 0.17% wt/wt, 0.18% wt/wt, 0.19% wt/wt, 0.20% wt/wt, 0.21% wt/wt, 0.22% wt/wt, 0.23% wt/wt, 0.24% wt/wt, 0.25% wt/wt, 0.26% wt/wt, 0.27% wt/wt, 0.28% wt/wt, 0.29% wt/wt, 0.30% wt/wt, while the total content of betamethasone or salt (e.g. betamethasone sodium phosphate) in the composition expressed as the mass concentration may be from 0.01% wt/wt to 0.08% wt/wt, such as 0.01%, 0.02%, 0.03%, 0.04%, 0.05%, 0.06%, 0.07%, and 0.08% wt/wt.

In some embodiments, any one or more of the compositions may also include one or more antioxidants selected from the group consisting of ascorbic acid or ascorbic acid derivatives, erythorbic acid, and sodium ascorbate; Thiol derivatives such as thioglycerol, cysteine, acetylcysteine, cystine, dithioerythreitol, dithiothreitol, glutathione; Tocopherols; butylated hydroxyanisol (BHA); butylated hydroxytoluene (BHT); sulfurous acid salts such as sodium sulfate, sodium bisulfite, acetone sodium bisulfite, sodium metabisulfite, sodium sulfite, sodium formaldehyde sulfoxylate, and sodium thiosulfate; and nordihydroguaiaretic acid.

The pharmaceutical compositions disclosed herein are preferably formulated for use as low viscosity eye drops for topical administration to the eye and thus include a pharmaceutically acceptable carrier that is acceptable for administration to the eye and does not increase viscosity of the formulation above about 25 cP. In certain embodiments, the carrier includes pure de-ionized water or a balanced salt solution. In yet other embodiments, the carrier may, in addition to water and/or a balanced salt solution, further optionally contain additional products, such as but not limited to those described below.

In some embodiments the carrier includes one or more lubricating agents, such as glycerol, glycerin, or glycerine. Alternatively, if desired, another compound may be used as a lubricating agent in addition to, or instead of, glycerol, glycerin, or glycerine, if desired. Non-limiting examples of acceptable lubricating agent(s) that may be so used include any of: polyvinyl pyrrolidone, sorbitol, polyethylene glycol, hydroxypropylmethyl cellulose, carboxy propylmethyl cellulose, and polyvinyl acetate. As generally guidance, lubricants can be provided at 0.1% wt/wt to 5.0% wt/wt, such as between 0.1% wt/wt and 1.0% wt/wt, for example 0.2% wt/wt.

Other products that may be used within the carrier include any excipient suitable for use in eye drop formulation. For example, the excipient can be a non-ionic polyoxyethlene-polyoxypropylene block copolymer having the following general structure:

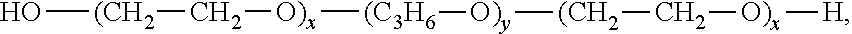

wherein x is an integer having the value of at least 8 and y is an integer having the value of at least 38.

If a non-ionic polyoxyethlene-polyoxypropylene block copolymer is used as an excipient, its contents in the overall composition may be from 0.01% wt/wt and 1.0% wt/wt, such as from 0.1% wt/wt to 1.0% wt/wt, for example, 0.2% wt/wt.

One non-limiting example of a specific non-ionic polyoxyethlene-polyoxypropylene block copolymer that can be used as a solubilizing and stabilizing agent in the pharmaceutical compositions of the instant invention is the product known under the trade name POLOXAMER 407 (poly(ethylene glycol)-block-poly(propylene glycol)-block-poly(ethylene glycol)), with the molecular weight of the polyoxypropylene portion of about 4,000 Daltons, about a 70% polyoxyethylene content, the overall molecular weight of between about 9,840 Daltons and about 14,600 Daltons.

Another type of product that can be used in the excipient portion of the pharmaceutical formulation may be water-soluble methylcellulose and hydroxypropyl methylcellulose polymers, such as METHOCEL family of products, for example, a hydroxypropyl methylcellulose product METHOCEL E4M. The compositions may also contain a quantity of preservative(s) such as benzalkonium chloride, if desired.

Yet another type of product that can be used in the excipient portion of the pharmaceutical formulation may be a polycarbophil polymer product (i.e., a polymeric product based on polyacrylic acid cross-linked with divinyl glycerol), which is available under a variety of trade names such as FIBERCON, EQUALACTIN, KONSYL FIVER, etc. If a polycarbophil product such as Noveon AA-1 is used, it may also be present as a part of mycophenolic acid solution, as mentioned above.

In addition, surfactants and/or anti-foaming agents may be provided within the carrier. As a non-limiting example, the carrier can include PLURONIC F-127

In another embodiment, the carrier includes the following: sodium thiosulfate, sodium chondroitin sulfate, Dextran-70, edetate disodium, poloxamer 407, glycerin, Hypromellose, potassium chloride, sodium phosphate dibasic anhydrous, sodium phosphate monobasic anhydrous, sodium hydroxide, hydrochloric acid, and sterile water.

The pharmaceutical compositions will typically have an osmolarity from 100 to 500 milliosmoles per liter (mOsm/L), such as from 150 mOsm/L to 450 mOsm/L, for example, from 200 mOsm/L to 400 mOsm/L. A tonicity modulating agent, such as sodium chloride, may also be used in the compositions.

The pharmaceutical compositions can be formulated using a one-batch method, where the components of the pharmaceutical formulation are combined in single container; though the components may be added to the container simultaneously or consecutively. Alternatively, a two- or multiple-batch method(s) may be used if desired, where each component of the pharmaceutical formulation is combined in a separate container followed by combining the contents of each container. The resulting product may then be transferred by sterile filtration into single dose vials, sealed, cooled, and pouched. Finally, a complete sterility and endotoxin analysis may be performed on the product according to commonly used methods known to those having ordinary skill in the art.

### B. Use of Pharmaceutical Compositions

Each of the above-described pharmaceutical compositions can be used for treating, preventing, and/or alleviating ocular disorders or signs and symptoms thereof, such as ocular surface disease, e.g., including, without limitation, keratoconjunctivitis sicca, episodic dry eye disease, chronic dry eye disease, recalcitrant dry eye disease, age-related dry eye, neurotrophic ocular surface disease, blepharitis, meibomian gland disease, ocular hyperemia, ocular allergy, corneal abrasion, STODS, discomfort from contact lens wear and others. The pharmaceutical compositions are also particularly useful for administration after ocular surgery (e.g. cataract surgery, refractive surgery and others), when such disorders or conditions are likely to occur. The compositions bring about a significant relief to the sufferers of such diseases. Among other benefits, the uncomfortable “stinging” or “burning” feeling in the eye that is routinely experienced in formulations including steroids is eliminated or at least significantly decreased after the composition has been administered. Furthermore, the compositions significantly reduce or eliminate the adverse effects commonly associated with long term, higher dosage use of steroids, such as increased intraocular pressure, which is a glaucoma risk factor, cataract, infection, and wound-healing delay. As such, unlike conventional steroid formulations, the compositions can be administered regularly (e.g. daily or twice daily) for extended periods of time, such as one (1) week, two (2) weeks, three (3) weeks, four (4) weeks or one (1) month, two (2) months, three (3) months, or more.

To this end, a method of treating an ocular disorder such as an ocular disease such as dry eye disease is provided, which includes: administering to a subject suffering from the ocular disorder, a therapeutically effective amount of a low viscosity pharmaceutical composition including a primary active ingredient(s) selected from the group consisting of betamethasone or a pharmaceutically acceptable salt thereof (e.g. betamethasone sodium phosphate) preferably at a concentration of 0.01% wt/wt to 0.08% wt/wt (e.g. 0.01%, 0.02%, 0.03%, 0.04%, 0.05%, 0.06%, 0.07%, 0.08% wt/wt or amounts therebetween) and/or mycophenolic acid or a pharmaceutically acceptable salt thereof (e.g. mycophenolate sodium) preferably at a concentration of 0.01% wt/wt to 0.30% wt/wt (e.g. 0.01% wt/wt, 0.02% wt/wt, 0.03% wt/wt, 0.4% wt/wt, 0.05% wt/wt, 0.06% wt/wt, 0.7% wt/wt, 0.8% wt/wt, 0.9% wt/wt, 0.10% wt/wt, 0.11% wt/wt, 0.12% wt/wt, 0.13% wt/wt 0.14% wt/wt, 0.15% wt/wt, 0.16% wt/wt, 0.17% wt/wt, 0.18% wt/wt, 0.19% wt/wt, 0.20% wt/wt, 0.21% wt/wt, 0.22% wt/wt, 0.23% wt/wt, 0.24% wt/wt, 0.25% wt/wt, 0.26% wt/wt, 0.27% wt/wt, 0.28% wt/wt, 0.29% wt/wt, 0.30% wt/wt, and amounts therebetween); each preferably including a glycosaminoglycan at 0.1% to 2.5% (e.g. 0.10% wt/wt, 0.11% wt/wt, 0.12% wt/wt, 0.13% wt/wt 0.14% wt/wt, 0.15% wt/wt, 0.16% wt/wt, 0.17% wt/wt, 0.18% wt/wt, 0.19% wt/wt, 0.20% wt/wt, 0.21% wt/wt, 0.22% wt/wt, 0.23% wt/wt, 0.24% wt/wt, 0.25% and amounts therebetween), and a pharmaceutically acceptable carrier for topical administration to the eye. These concentration guidelines are based on amounts of betamethasone sodium phosphate. One having ordinary skill in the art to which the invention belongs will appreciate that the concentration of betamethasone could differ by as much as about 25% in the formulations since betamethasone has a molecular weight that is about 25% less than betamethasone sodium phosphate (betamethasone sodium phosphate has a molecular weight of 516 compared to a molecular weight of 392 for betamethasone). In some embodiments, the mycophenolic acid or pharmaceutically acceptable salt is at a concentration of 0.05% wt/wt to 0.30% wt/wt. These concentration guidelines are based on amounts of mycophenolic acid. When using a salt such as mycophenolate or mycophenolate sodium, the amounts can be adjusted so that the mycophenolic acid is present at the described ranges. In some embodiments, the pharmaceutical composition of the betamethasone or salt, and/or the mycophenolic acid or salt and the pharmaceutically acceptable carrier, preferably include a lubricant, for topical administration to the eye. Despite the low viscosity, the pharmaceutical composition includes one or more of a glycosaminoglycan (e.g. chondroitin sulfate from 0.1% to 2.5% wt/wt); a deturgescent agent (e.g. dextran from 0.1% to 5.0% wt/wt); and other compounds substantially as described above or below or as known in the art to which the invention belongs. Preferably, the pharmaceutical composition is administered dropwise (e.g. one to two drops per eye, twice daily) over one or more weeks, such as one week, two weeks, three weeks, four weeks or one month, two months, three months or more.

In furtherance of the above, the following pharmaceutical compositions were tested against 0.05% cyclosporine ophthalmic emulsion (RESTASIS) and 5% lifitegrast ophthalmic solution (XIIDRA) as a treatment for dry eye disease: a composition containing 0.01% betamethasone sodium phosphate and 0.3% mycophenolic acid (SURF-100), compositions containing either 0.1% or 0.3% mycophenolic acid, and 0.01% betamethasone sodium phosphate. Subjects were dosed twice daily (BID) for 84 days (12 weeks). The study is provided in more detail in Example 7 and exemplary results showing the first 30 days of treatment are provided in FIGS. 1-3. In summary, the mycophenolic acid and/or betamethasone sodium phosphate formulations were highly effective at reducing dry eye signs and symptoms and subjects did not report adverse events associated with administration of higher doses of steroids.

In instances where the subject is suffering from severe inflammation, the betamethasone sodium phosphate can be temporarily increased, such as to 0.2% wt/wt or about 0.2% wt/wt with or without the mycophenolic acid at 0.01% wt/wt to 0.5% wt/wt, for administration over short time frames (e.g. several days to 2 weeks maximum with minimal dosing per day). Preferably, this formulation also includes chondroitin sulfate at about 0.1 to 2.5% wt/wt.

In related embodiments, a post-surgical ocular treatment method is also provided, which includes topically administering to an eye of a subject that has undergone ocular surgery, a low viscosity pharmaceutical composition including or consisting essentially of betamethasone or a pharmaceutically acceptable salt thereof at a concentration of 0.2% wt/wt for no more than 14 consecutive days; and a glycosaminoglycan. In some embodiment the pharmaceutical composition also includes mycophenolic acid or a pharmaceutically acceptable salt thereof at a concentration of 0.05% wt/wt/to 0.30% wt/wt. In some embodiments, the method includes administering a second low viscosity pharmaceutical composition including mycophenolic acid or a pharmaceutically acceptable salt thereof at a concentration of 0.05% wt/wt to 0.30% wt/wt after the no more than 14 days; optionally betamethasone or a pharmaceutically acceptable salt thereof at 0.01% wt/wt to 0.08% wt/wt; and a same or different glycosaminoglycan. The second treatment can be administered regularly (e.g. one to three times daily) over periods of between one week and many months, such as one week, two weeks, three weeks, four weeks or one month, two months, three months, or more. These concentration guidelines are based on amounts of betamethasone sodium phosphate. One having ordinary skill in the art to which the invention belongs will appreciate that the concentration of betamethasone could differ by as much as about 25% in the formulations since betamethasone has a molecular weight that is about 25% less than betamethasone sodium phosphate (betamethasone sodium phosphate has a molecular weight of 516 compared to a molecular weight of 392 for betamethasone).

In still another related embodiment, a method of treating hyperemia of the eye is provided, which includes administering to an eye of a subject suffering from hyperemia, a therapeutically effective amount of a low viscosity pharmaceutical composition including mycophenolic acid or pharmaceutically acceptable salt thereof (e.g. mycophenolate sodium), betamethasone or a pharmaceutically acceptable salt thereof (e.g. betamethasone sodium phosphate), or a combination of mycophenolic acid and betamethasone or their pharmaceutically acceptable salts; each with a glycosaminoglycan. Conjunctive hyperemia is a common clinical ophthalmological finding and can be a sign of various ocular disorders, such as conjunctivitis, uveitis, dry eye, allergies, elevated intraocular pressure due to glaucoma, and ophthalmic side effects. Thus, treating conjunctival hyperemia can simultaneously treat an underlying disorder selected from conjunctivitis, uveitis, dry eye, allergies, elevated intraocular pressure due to glaucoma, and others. As demonstrated in FIG. 1, 0.3% mycophenolic acid (MPA) and 0.01% betamethasone sodium phosphate (BSP), each administered alone (but with a chondroitin sulfate-based carrier) was effective at treating subjects suffering from hyperemia of the eye; although over the first week, BSP worked better than MPA. However, during the study, it was surprisingly revealed that the combination of 0.3% MPA and 0.01% BSP worked significantly better than the MPA and BSP alone. It was also surprising to find that the combined treatment showed significant improvement almost immediately and maintained this significant improvement over at least the first month.

When compared against XIIDRA and RESTASIS, reduction in conjunctival hyperemia was more effective in subjects treated with MPA and BSP individually and when treated with MPA/BSP in combination, each MPA/BSP formulation with chondroitin sulfate. Both 0.3% MPA and 0.01% BSP alone and in combination (SURF-100), performed better than XIIDRA and RESTASIS. However, it was also surprisingly found that 0.1% MPA was the most effective. This was surprising because it is at a significantly lower dose than previously tested. From this, it is now believed that combining 0.01%-0.30% MPA (such as 0.05% to 0.30%) and BSP, such as 0.01% to 0.08% BSP, may provide a still better treatment for hyperemia.

It was also found that conjunctival hyperemia could be significantly reduced over long periods of time after one receiving a pulsed administration of a low dose of betamethasone sodium phosphate. FIG. 5 shows that administering betamethasone sodium phosphate at 0.02% wt/wt or 0.04% wt/wt twice daily for only two weeks can significantly reduce conjunctival hyperemia over a period of at least 70 days.

These concentration guidelines are based on amounts of betamethasone sodium phosphate. One having ordinary skill in the art to which the invention belongs will appreciate that the concentration of betamethasone could differ by as much as about 25% in the formulations since betamethasone has a molecular weight that is about 25% less than betamethasone sodium phosphate (betamethasone sodium phosphate has a molecular weight of 516 compared to a molecular weight of 392 for betamethasone).

In still another related embodiment, a method of treating uveitis is provided, which includes administering to an eye of a subject suffering from uveitis, a therapeutically effective amount of a low viscosity pharmaceutical composition including or consisting essentially of a primary active ingredient including mycophenolic acid or a pharmaceutically acceptable salt thereof (e.g. mycophenolate sodium), betamethasone or a pharmaceutically acceptable salt thereof (e.g. betamethasone sodium phosphate), or a combination of mycophenolic acid and betamethasone or their salts; each with a glycosaminoglycan, preferably chondroitin sulfate. Uveitis is an inflammation inside the eye. The uvea is the middle layer of the eye between the sclera (white part of the eyes) and the retina (light-sensitive layer at the back of the eye). Uveitis can occur when the body is fighting an infection of the eye, although it can also happen with the immune system attacks healthy tissue in the eyes. Uveitis can cause pain, redness, and vision loss.

In some embodiments, treatment for uveitis can be by way of administering the low viscosity pharmaceutical composition of 0.05% to 0.30% wt/wt mycophenolic acid or salt and/or 0.01% to 0.08% betamethasone or salt; each with a glycosaminoglycan, preferably chondroitin sulfate. Administration can be topically or by injection. Administration can be performed regularly (e.g. one to three times daily) over periods of between one week and many months, such as one week, two weeks, three weeks, four weeks or one month, two months, three months, or more. In some embodiments, treatment is performed regularly until signs and/or symptoms subside. In instances where discomfort is extreme, temporary use of a betamethasone sodium phosphate solution at 0.2% wt/wt may interrupt administration. These concentration guidelines are based on amounts of betamethasone sodium phosphate. One having ordinary skill in the art to which the invention belongs will appreciate that the concentration of betamethasone could differ by as much as about 25% in the formulations since betamethasone has a molecular weight that is about 25% less than betamethasone sodium phosphate (betamethasone sodium phosphate has a molecular weight of 516 compared to a molecular weight of 392 for betamethasone). In addition, these concentration guidelines are based on amounts of mycophenolic acid. When using a salt such as mycophenolate or mycophenolate sodium, the amounts can be adjusted so that the mycophenolic acid is present at the described ranges.

In still another related embodiment, a method of conjunctive epithelial cell healing is provided, which includes administering to the eyes of a subject suffering from damaged or diseased conjunctival epithelium, a therapeutically effective amount of a low viscosity pharmaceutical composition, the pharmaceutical composition including or consisting essentially of a primary active ingredient(s) selected from the group consisting of mycophenolic acid or a pharmaceutically acceptable salt thereof (e.g. mycophenolate sodium), betamethasone or a pharmaceutically acceptable salt thereof (e.g. betamethasone sodium phosphate), or a combination of mycophenolic acid and betamethasone or their pharmaceutically acceptable salts thereof; each with a glycosaminoglycan, preferably chondroitin sulfate. The conjunctiva is a translucent membrane that lines the inner surface of the eyelids and covers the sclera. It is overlayed by two layers of stratified, non-keratinized conjunctival epithelial cells. These cells are covered by a mucin-rich glycocalyx, which promotes tear adherence, prevents pathogen penetration, and provides lubrication. Defective glycocalyx production or maintenance can lead to ocular surface disease and dry eye. Without being bound by theory, it is believed that administering the pharmaceutical composition improves tear adherence and improves lubrication by acting at least in part on the conjunctival epithelium.

In some embodiments, treating the conjunctive epithelium includes administering the low viscosity pharmaceutical composition of 0.05%-0.30% wt/wt mycophenolic acid or salt (e.g. mycophenolate sodium) and/or 0.01%-0.08% betamethasone or salt (e.g. betamethasone sodium phosphate); each with a glycosaminoglycan, preferably chondroitin sulfate. Administration can be topically or by injection. Administration can be performed regularly (e.g. daily, twice daily) over one or more weeks, such as one week, two weeks, three weeks, four weeks or one month, two months, three months or more. In some embodiments, treatment is performed regularly until signs and/or symptoms subside. These concentration guidelines are based on amounts of betamethasone sodium phosphate. One having ordinary skill in the art to which the invention belongs will appreciate that the concentration of betamethasone could differ by as much as about 25% in the formulations since betamethasone has a molecular weight that is about 25% less than betamethasone sodium phosphate (betamethasone sodium phosphate has a molecular weight of 516 compared to a molecular weight of 392 for betamethasone). In addition, these concentration guidelines are based on amounts of mycophenolic acid. When using a salt such as mycophenolate or mycophenolate sodium, the amounts can be adjusted so that the mycophenolic acid is present at the described ranges

In still other embodiments, the low viscosity pharmaceutical compositions are used in corneal transplant procedures. Corneal transplant is a procedure that removes all or part of a damaged cornea and replaces it with healthy donor tissue. Corneal transplants can improve sight and relieve pain. After undergoing corneal transplant, patients are typically given eyedrops to help the eye heal and prevent the body from rejecting the donor tissue. The pharmaceutical compositions described herein have been shown to reduce conjunctival hyperemia and will have therapeutic utility in subjects that undergo corneal transplant. As shown in FIGS. 13, and 5, the pharmaceutical compositions decrease hyperemia in patients suffering from ocular conditions and as shown in FIGS. 2 and 4, subject's pain and discomfort when suffering from ocular conditions is significantly reduced when administering mycophenolic acid, betamethasone sodium phosphate, or mycophenolic acid and betamethasone sodium phosphate; each with a glycosaminoglycan, preferably chondroitin sulfate. Accordingly, use of the pharmaceutical compositions in a corneal transplant procedure is provided, which includes administering to an eye of a subject, before, during and/or after a corneal transplant procedure, a therapeutically effective amount of a pharmaceutical composition including or consisting essentially of a primary active ingredient(s) selected from the group consisting of mycophenolic acid or a pharmaceutically acceptable salt thereof (e.g. mycophenolate sodium), betamethasone or a pharmaceutically acceptable salt thereof (e.g. betamethasone sodium phosphate), or a combination of both mycophenolic acid and betamethasone or their pharmaceutically acceptable salts; each with a glycosaminoglycan, preferably chondroitin sulfate.

When used in a corneal transplant procedure, in some embodiments, the low viscosity pharmaceutical composition includes or consists essentially of 0.05%-0.30% wt/wt mycophenolic acid or salt (e.g. mycophenolate sodium) and/or 0.01%-0.08% betamethasone or salt (e.g. betamethasone sodium phosphate); each with a glycosaminoglycan, preferably chondroitin sulfate. Administration can be topically, such as in the form of eye drops before or after the procedure, in a rinse step during the procedure or by injection before or during the procedure. In some embodiments, the pharmaceutical composition is provided during the procedure as a therapeutic rinse, followed by in the form of eye drops as a post operative treatment. Depending on the stage administration, it can be performed once or regularly (e.g. one to three times daily) over periods of between one week and many months, such as one week, two weeks, three weeks, four weeks or one month, two months, three months, or more. In some embodiments, treatment is performed regularly after the procedure until signs and/or symptoms subside. These concentration guidelines are based on amounts of betamethasone sodium phosphate. One having ordinary skill in the art to which the invention belongs will appreciate that the concentration of betamethasone could differ by as much as about 25% in the formulations since betamethasone has a molecular weight that is about 25% less than betamethasone sodium phosphate (betamethasone sodium phosphate has a molecular weight of 516 compared to a molecular weight of 392 for betamethasone). In addition, these concentration guidelines are based on amounts of mycophenolic acid. When using a salt such as mycophenolate or mycophenolate sodium, the amounts can be adjusted so that the mycophenolic acid is present at the described ranges

Still further, the pharmaceutical compositions described hereinabove may be useful for preventative and therapeutic treatment of other ophthalmic conditions and diseases as they are expected to provide numerous medical benefits such as for ocular surface (e.g., cornea and conjunctiva) lubrication, corneal deturgescence, cell membrane stabilization, etc. The pharmaceutical compositions described hereinabove may be further useful for protecting the ocular surface, corneal epithelial cells, corneal endothelial cells, and/or other ocular tissues during an eye surgery. In addition, the ophthalmic compositions may be useful in wound healing after various injuries to the eye, for reducing corneal edema (e.g., during and after corneal transplantation surgery), for rehabilitating the ocular surface before and after contact lens wear, etc.

Pharmaceutical formulations described herein are preferably delivered topically, e.g., via eye drops. An ordinarily skilled physician may prescribe delivery by any other acceptable method if so desired and indicated.

In each of the described uses, the pharmaceutical compositions described hereinabove may be provided as a single dosage, in periodic applications, or may be maintained on the ophthalmic tissue continuously or substantially continuously as appropriate for the particular use. For example, they may be administered as a single drop, twice per day (BID), once per day, or once every minute for a period of 5 to 10 minutes, or more frequently, or less frequently. Clinical testing of the pharmaceutical compositions suggests twice per day (BID) may be preferred; however, it is envisioned that the pharmaceutical composition can be applied between 1 to 16 times a day (e.g., from 1 to 8 times per day, from 1 to 6 times per day, from 1 to 4 times per day or from 1 to 2 times per day), or more frequently, or less frequently, as needed. At least in part due to the low concentration of the steroid component, treatment periods can be extended to weeks or months without the risks associated with conventional therapeutics, e.g. glaucoma, cataract, infection, and wound-healing delay.

Though administering betamethasone sodium phosphate and mycophenolic acid at the above listed low concentrations, twice daily, provides a benefit over conventional therapeutics, it will be understood by those having ordinary skill in the art that the specific dose level and frequency of dosage for any particular patient may be varied and will depend upon a variety of factors including the activity of the specific compound employed, the metabolic stability and length of action of that compound, the age, body weight, general health, gender, diet, and the severity of the particular disease or condition being treated.

### C. EXAMPLES

The following examples are provided to further elucidate the advantages and features of the present invention but are not intended to limit the scope of the invention. The examples are for the illustrative purposes only. USP pharmaceutical grade products were used in preparing the formulations described below.

### Example 1. Preparing Pharmaceutical Composition Nos. 1 and 2

Pharmaceutical compositions were prepared as described below. The following products were used in the amounts specified:
(a) 0.107 (Composition 1) or 0.321 (Composition 2) of mycophenolate sodium powder;
(b) 0.25 g of chondroitin sulfate (bovine);
(c) 0.25 of powdered dextran-70;
(d) 0.1 g of edetate disodium powder;
(e) 0.30 g of powdered sodium thiosulfate pentahydrate;
(f) 0.03 g potassium chloride;
(g) 0.20 g of PLURONIC® F-127;
(h) 0.2 g of glycerol;
(i) 0.9 g of sodium phosphate dibasic anhydrous;
(j) 0.18 g of sodium phosphate monobasic anhydrous;
(k) 100 ml of sterile injectable water.

Chondroitin sulfate, dextran, sodium thiosulfate, potassium phosphate, dibasic and monobasic sodium phosphate, potassium chloride, EDTA, and PLURONIC® F-127 were combined with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium was added slowly and adjusting pH to about 7.0-7.4 and the remainder of water was added. Additional, sodium hydroxide may be added after the mycophenolate sodium to obtain the final pH, if necessary. The solution was then filtered through a 0.2 micron filter into a sterile droptainer.

### Example 2. Preparing Pharmaceutical Composition Nos. 3 and 4

Pharmaceutical compositions were prepared as described below. The following products were used in the amounts specified:
(a) 0.02 (Composition 3) or 0.04 g (Composition 4) of betamethasone sodium phosphate;
(b) 0.25 g of chondroitin sulfate (bovine);
(c) 0.25 of powdered dextran-70;
(d) 0.1 g of edetate disodium powder;
(e) 0.30 g of powdered sodium thiosulfate pentahydrate;
(f) 0.03 g potassium chloride;
(g) 0.20 g of PLURONIC® F-127;
(h) 0.2 g of glycerol;
(i) 0.9 g of sodium phosphate dibasic anhydrous;
(j) 0.18 g of sodium phosphate monobasic anhydrous;
(k) 100 ml of sterile injectable water.

Chondroitin sulfate, dextran, sodium thiosulfate, phosphate buffer, potassium chloride, EDTA, and PLURONIC® F-127 were combined with about 90% of the water and stirred until completely dissolved followed by addition of glycerol with continued stirring. The pH of the solution was then adjusted to about 7.0 using sodium hydroxide solution before introducing mycophenolate sodium. A stock solution of 1% betamethasone sodium phosphate was prepared by dissolving 1 μm of betamethasone sodium phosphate powder in 100 ml water and confirming the stock solution concentration by HPLC. 1 ml of the 1% betamethasone sodium phosphate stock solution was then added to the mixture with continued stirring.

With continued stirring sodium hydroxide adjusting pH to about 7.3-7.4, and addition of the remainder of the water. The solution was then filtered through a 0.2 micron filter into sterile dropper bottles or unit dose vials.

### Example 3. Preparing a Pharmaceutical Composition Nos. 5 and 6

Pharmaceutical compositions were prepared as described below. The following products were used in the amounts specified:
(a) 0.01 g of betamethasone sodium phosphate;
(b) 0.107 g (Composition 5) or 0.321 g (Composition 6) mycophenolate sodium;
(c) 0.25 g of chondroitin sulfate (bovine);
(d) 0.25 g of powdered dextran-70;
(e) 0.1 g of edetate disodium powder;
(f) 0.30 g of powdered sodium thiosulfate pentahydrate;
(g) 0.03 g potassium chloride;
(h) 0.20 g of PLURONIC® F-127;
(i) 0.2 g of glycerol;
(j) 0.9 g of sodium phosphate dibasic anhydrous;
(k) 0.18 g of sodium phosphate monobasic anhydrous;
(l) 100 ml of sterile injectable water.

Chondroitin sulfate, dextran, sodium thiosulfate, potassium chloride, EDTA, phosphate buffer, and PLURONIC® F-127 were combined with about 90% of the water and stirred until completely dissolved followed by addition of glycerol with continued stirring. The pH of the solution was then adjusted to about 7.0 using sodium hydroxide solution before introducing mycophenolate sodium. A stock solution of 1% betamethasone sodium phosphate was prepared by dissolving 1 μm of betamethasone sodium phosphate powder in 100 ml water and confirming the stock solution concentration by HPLC. 1 ml of the 1% betamethasone sodium phosphate stock solution was then added to the mixture with continued stirring.

With continued stirring, mycophenolate sodium was added slowly followed by addition of sodium hydroxide to a pH of about 7.3-7.4, and addition of the remainder of the water. The solution was then filtered through a 0.2 micron filter into sterile dropper bottles or unit dose vials.

### Example 4. Preparing Pharmaceutical Composition No. 7

A pharmaceutical composition was prepared as described below. The following products were used in the amounts specified:
(a) 0.0107 g of mycophenolate sodium;
(b) 0.25 g of chondroitin sulfate (bovine);
(c) 0.25 g of powdered dextran-70;
(d) 0.1 g of edetate disodium dihydrate;
(e) 0.2 g of PLURONIC® F-127;
(f) 0.2 g of glycerin;
(g) 0.30 g of powdered sodium thiosulfate pentahydrate;
(h) 0.03 g of potassium chloride;
(i) 0.9 g of sodium phosphate dibasic anhydrous;
(j) 0.18 g of sodium phosphate monobasic anhydrous;
(k) 100 ml of sterile injectable water.

Chondroitin sulfate, dextran, EDTA, sodium thiosulfate, glycerol, potassium chloride, potassium phosphate, dibasic and monobasic sodium phosphate and PLURONIC® F-127 were combined with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium was added slowly and adjusting pH to about 7.0-7.4 and the remainder of water was added. Additional, sodium hydroxide may be added after the mycophenolate sodium to obtain the final pH, if necessary. The solution was then filtered through a 0.2 micron filter into a sterile droptainer.

### Example 5: Preparing a Pharmaceutical Composition No. 8

A pharmaceutical composition was prepared as described below. The following products were used in the amounts specified:
(a) 0.0214 g of Mycophenolate Sodium;
(b) 0.25 g of chondroitin sulfate (bovine);
(c) 0.25 g of powdered dextran-70;
(d) 0.1 g of edetate disodium dihydrate;
(e) 0.2 g of PLURONIC® F-127;
(f) 0.2 g of glycerin;
(g) 0.30 g of powdered sodium thiosulfate pentahydrate;
(h) 0.03 g of potassium chloride;
(i) 0.9 g of sodium phosphate dibasic anhydrous;
(j) 0.18 g of sodium phosphate monobasic anhydrous;
(k) 100 ml of sterile injectable water

Chondroitin sulfate, dextran, EDTA, sodium thiosulfate, glycerol, potassium chloride, potassium phosphate, dibasic and monobasic sodium phosphate and PLURONIC® F-127 were combined with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium was added slowly and adjusting pH to about 7.0-7.4 and the remainder of water was added. Additional, sodium hydroxide may be added after the mycophenolate sodium to obtain the final pH, if necessary. The solution was then filtered through a 0.2 micron filter into a sterile droptainer.

### Example 6: Preparing a Pharmaceutical Composition No. 9

A pharmaceutical composition was prepared as described below. The following products were used in the amounts specified:
(a) 0.0537 g of Mycophenolate Sodium;
(b) 0.25 g of chondroitin sulfate (bovine);
(c) 0.25 g of powdered dextran-70;
(d) 0.1 g of edetate disodium dihydrate;
(e) 0.2 g of PLURONIC® F-127;
(f) 0.2 g of glycerin;
(g) 0.30 g of powdered sodium thiosulfate pentahydrate;
(h) 0.03 g of potassium chloride;
(i) 0.9 g of sodium phosphate dibasic anhydrous;
(j) 0.18 g of sodium phosphate monobasic anhydrous;
(k) 100 ml of sterile injectable water.

Chondroitin sulfate, dextran, EDTA, sodium thiosulfate, glycerol, potassium chloride, potassium phosphate, dibasic and monobasic sodium phosphate and PLURONIC® F-127 were combined with about 90% of water and stirred until completely dissolved followed by adding glycerol with continued stirring. The pH of the solution was then adjusted to about 7.0 using sodium hydroxide solution before introducing mycophenolate sodium.

With continued stirring, mycophenolate sodium was added slowly and adjusting pH to about 7.0-7.4 and the remainder of water was added. Additional, sodium hydroxide may be added after the mycophenolate sodium to obtain the final pH, if necessary. The solution was then filtered through a 0.2 micron filter into a sterile droptainer.

### Example 7. Use of Mycophenolic Acid and Betamethasone Sodium Phosphate for the Treatment of Ocular Disorders

Combinations of mycophenolic acid (MPA) and betamethasone sodium phosphate (BSP) were evaluated for ocular safety, tolerability, and efficacy. Among these, a combination of 0.3% MPA and 0.01% BSP (also referred to as SURF-100) was compared to 0.1% MPA; 0.3% MPA; 0.01% BSP; vehicle control; 0.05% cyclosporine ophthalmic emulsion (RESTASIS); and 5% lifitegrast ophthalmic solution (XIIDRA). The vehicle control includes the following: sodium thiosulfate, sodium chondroitin sulfate, Dextran-70, edetate disodium, poloxamer 407, glycerin, potassium chloride, sodium phosphate dibasic anhydrous, sodium phosphate monobasic anhydrous, sodium hydroxide, hydrochloric acid, and sterile water. The subjects were adult subjects with dry eye disease, the compositions are dosed twice daily (once in the morning and once in the evening) for up to 12 weeks, and the subjects followed up with about every 2 weeks post-dosing.

Before beginning, during the dosing period, and in follow up visits, the following assessments were performed: best corrected visual acuity (BCVA), intraocular pressure (IOP), slit lamp biomicroscopy, ophthalmoscopy, fluorescein corneal staining, conjunctival staining, conjunctival hyperemia, UNC DEMS questionnaire, tear break-up time (TBUT), and Schirmer tear test score. In addition, assessment for adverse events was also performed at visits.

FIG. 1 depicts results from the conjunctival hyperemia testing. In the graph, the legend symbols are used solely to distinguish the treatment timelines from one another. Conjunctival hyperemia is caused by a pathological vasodilatory response of the microvasculature in response to inflammation due to a myriad of infection and non-infectious etiologies. While the underlying etiology of ocular surface diseases can be difficult to determine on a case-by-case basis, the ocular hyperemia appearing due to the condition is consistent and therefore provides an effective measurement to assess potential treatments. As shown in FIG. 1, the combination of 0.3% MPA and 0.01% BSP provided significantly reduced conjunctival hyperemia compared to 0.3% MPA or 0.01% BSP alone. In particular, the combined treatment resulted in a surprising decrease in conjunctival hyperemia over the first 30 days and showed significant improvement immediately after treatment. In contrast, during the first one to three weeks of treatment with either MPA or BSP alone, the decrease in hyperemia was the same or about the same and thus each showed approximately the same efficacy over longer periods of time.

UNC-DEMS is another commonly accepted approach to assess dry eye status and treatment effectiveness. Under UNC-DEMS, subjects are asked to rate symptoms and effects encountered on daily life from 1 to 10. As shown in FIG. 2, the results coincide with the results depicted in FIG. 1. In the graph, the legend symbols are used solely to distinguish the treatment timelines from one another. Here again, the combination of 0.3% MPA and 0.01% BSP provided significantly more relief compared to 0.3% MPA and 0.01% BSP alone. In particular, the combined treatment resulted in a surprising decrease in patient relief over the first two and half months and showed significant improvement immediately after treatment. Both 0.3% MPA and 0.01% BSP individually were about equally as effective to one another and substantially less effective than the combined treatment.

FIG. 3 depicts results from conjunctival hyperemia testing comparing 0.3% MPA and 0.01% BSP (SURF-100) to 0.1% MPA; 0.3% MPA; 0.01% BSP; vehicle control; 0.05% cyclosporine ophthalmic emulsion (RESTASIS); and 5% lifitegrast ophthalmic solution (XIIDRA). In the graph, the legend symbols are used solely to distinguish the treatment timelines from one another. Here, SURF-100 was far superior to 0.3% MPA; 0.01% BSP; Vehicle; RESTASIS; and XIIDRA over the first month of treatment. However, it was also surprisingly found that 0.1% MPA provided even significantly better conjunctival hyperemia scores, which suggests its use at dosages at least as low as 0.1% or 0.05% MPA.

### Example 8. Effect of a Shortened Dosage Regimen of Betamethasone Sodium Phosphate in the Treatment of Ocular Disorders

To assess the efficacy of short-term dosing of betamethasone sodium phosphate (BSP) on the treatment of ocular disorders, subjects with dry eye disease were administered either 0.02% BSP or 0.04% BSP twice daily (once in the morning and once in the evening) for 2 weeks, and follow ups were conducted over an additional two months.

Before beginning, during the two-week dosing period, and in the follow ups, the following assessments are performed: best corrected visual acuity (BCVA), intraocular pressure (IOP), slit lamp biomicroscopy, ophthalmoscopy, fluorescein corneal staining, conjunctival staining, conjunctival hyperemia, UNC DEMS questionnaire, tear break-up time (TBUT), and Schirmer tear test score. In addition, assessment for adverse events was also performed.

FIG. 4 depicts results from UNC-DEMS questionnaire regarding symptoms over the 70 day period, where subjects were asked to rate symptoms and effects encountered on daily life from 1 to 10. In the graph, the legend symbols are used solely to distinguish the treatment timelines from one another. Significant improvement was reported in both treatment groups. Interestingly, subjects treated with the higher dose reported greater improvement initially, but subjects treated with the lower dose seemed to show slightly better improvement compared to those treated with the higher dose over the longer term. Both maintained significant improvement in UNC-DEMS scoring over the entirety of the follow-up period.

FIG. 5 depicts results from conjunctival hyperemia testing. In the graph, the legend symbols are used solely to distinguish the treatment timelines from one another. FIG. 5 shows that both 0.02% BSP and 0.04% BSP treatment groups showed significant improvement in conjunctival hyperemia over the entire 70-day test period with the higher concentration being more effective initially.

FIG. 6 depicts results from Anesthetized Shirmer Testing, which is used to quantify tear production for dry eye disease. In the graph, the legend symbols are used solely to distinguish the treatment timelines from one another. Broadly, the test is performed by placing filter paper inside the lower lid of the eye. After 5 minutes, the paper is removed and tested for moisture content. This change in tear production was tracked over time. Improvement in tear production was found in both groups of tested subjects over the entire testing period. Subjects treated with 0.04% BSP showed greater improvement in tear production initially compared to subjects treated with 0.02% BSP, but subjects treated with the lower dose showed greater improvement in tear production over the longer term.

FIG. 7 depicts results from tear break-up time (TBUT) analysis. In the graph, the legend symbols are used solely to distinguish the treatment timelines from one another. TBUT is a clinical test used to monitor tear film evaporation. To measure TBUT, fluorescein (a fluorescent compound) is instilled into the subject's tear film and the subject is asked not to blink while the tear film is observed under a broad beam of cobalt blue illumination. The TBUT is recorded as the number of seconds that elapse between the last blink and the appearance of the first dry spot in the tear film. A TBUT under 10 seconds is considered abnormal. Here, TBUT was measured and compared to the initial time to assess any change. Both 0.02% and 0.04% BSP were successful at reducing evaporation and thus improving the quality of the tear film, although better results were achieved when using the higher dose of BSP.

Although the invention has been described with the reference to the above examples, it will be understood that modifications and variations are encompassed within the spirit and scope of the invention. Accordingly, the invention is limited only by the following claims.

### REFERENCES CITED

AAO (2018). Dry eye syndrome. Preferred Practice Pattern. American Academy of Ophthalmology (AAO).
Baltatzis S1, Tufail F, Yu E N, Vredeveld C M, Foster C S. Mycophenolate mofetil as an immunomodulatory agent in the treatment of chronic ocular inflammatory disorders. Ophthalmology. 2003; 110 (5): 1061-5.
Baudouin C, Aragona P, Van Setten G et al. Diagnosing the severity of dry eye: a clear and practical algorithm. Br J Ophthalmol. 2014; 98 (9): 1168-1176. DOI: 10.1136/bjophthalmol-2013-304619.
Craig J P, Nelson J D, Azar D T et al. TFOS DEWS Il report executive summary. The Ocular Surface. 2017a; 15:802-812.
Ervin A, Law A et al. Punctal occlusion for dry eye syndrome. Cochrane Database Syst Rev. 2018; 6: CD006775. doi: 10.1002/14651858.CD006775.pub3.
Hyon J Y, Kim H-M, Lee D et al. Korean guidelines for the diagnosis and management of dry eye: development and validation of clinical efficacy. Korean J Ophthalmol. 2014; 28 (3): 197-206.
Omnipred PI (2006). Prednisolone acetate ophthalmic suspension prescribing information. NDA 17-469/S-040.
PredForte PI (2018). Prednisolone acetate ophthalmic suspension, USP 1% prescribing information. Allergan.
RESTASIS PI (2017). RESTASIS® (cyclosporine ophthalmic emulsion) 0.05% for topical ophthalmic use highlights of prescribing information (PI). Initial U.S. Approval: 1983. Allergan, Irvine CA.
Stern M E, Gao J, Siemasko K F, Beuerman R W, Pflugfelder SC. The role of the lacrimal functional unit in the pathophysiology of dry eye. Exp Eye Res. 2004; 78:409-416.
Willeke P, Schlüter B, Becker H, Schotte H, Domschke W, Gaubitz M. Mycophenolate sodium treatment in patients with primary Sjögren syndrome: a pilot trial. Arthritis Research & Therapy. 2007; 9: R115. DOI: 10.1186/ar2322.
XIIDRA PI (2016). XIIDRA (lifitegrast ophthalmic solution) 5%, for topical ophthalmic use. Initial U.S. Approval: 2016.

What is claimed is:

1. A pharmaceutical composition for topical administration to an eye, the composition comprising:
a. mycophenolic acid or a pharmaceutically acceptable salt thereof at a concentration of 0.05% to 0.3% wt/wt;
b. a glucosaminoglycan at a concentration of 0.1% to 2.5% wt/wt; and
c. a viscosity of 1 to 25 centipoise (cp).

2. The pharmaceutical composition of claim 1, wherein the glucosaminoglycan is chondroitin sulfate.

3. The pharmaceutical composition of claim 1, wherein the pharmaceutical composition is free of hyaluronic acid and glucosamine sulfate.

4. The pharmaceutical composition of claim 1, wherein the viscosity is 1 to 10 cp.

5. The pharmaceutical composition of claim 1, further comprising a betamethasone or a pharmaceutically acceptable salt thereof at a concentration of 0.01% to 0.08% wt/wt.

6. A method of treating ocular pain or discomfort, the method comprising topically administering to an eye of a subject suffering from ocular pain or discomfort, a therapeutically effective amount of the composition of claim 1.

7. The method of claim 6, wherein the ocular pain or discomfort is from a condition selected from the group consisting of an eye allergy, a corneal abrasion, contact lens wear, STODS, ocular surgery, and an ocular surface disease.

8. The method of claim 7, wherein the ocular surgery is cataract surgery or refractive surgery.

9. The method of claim 7, wherein the ocular surface disease is selected from the group consisting of dry eye disease, contact lens induced ocular surface dry eye disease, and preservative induced ocular surface disease.

10. The method of claim 6, wherein the pharmaceutical composition is administered for 7 consecutive days.

11. The method of claim 10, wherein the pharmaceutical composition is administered for 14 or fewer consecutive days.

12. A post-surgical ocular treatment method, comprising topically administering to an eye of a subject that has undergone ocular surgery, the pharmaceutical composition of claim 1.

13. The method of claim 12, wherein the pharmaceutical composition is administered for 7 consecutive days.

14. The method of claim 13, wherein the pharmaceutical composition is administered for 14 or fewer consecutive days.
